(19) United States
(12) Patent Application Publication
(10) Pub. No.: US 2008/0311552 A1
(43) Pub. Date: Dec. 18, 2008

(54) Use of Sirnas in Organ Storage/Reperfusion Solutions

(75) Inventor: Weiping Min, London (CA)

Correspondence Address: KNOBBE MARTENS OLSON & BEAR LLP, 2040 MAIN STREET, FOURTEENTH FLOOR, IRVINE, CA, 92614, US

(73) Assignee: London Health Sciences Centre Research, Inc., London, Ontario (CA)

(21) Appl. No.: 12/067,542

(22) PCT Filed: Sep. 20, 2006
(86) PCT No.: PCT/CA2006/001543

§ 371 Date: Aug. 14, 2008

Related U.S. Application Data
(60) Provisional application No. 60/718,327, filed on Sep. 20, 2005.

(51) Int. Cl. A01N 1/02; C12N 5/06

(52) U.S. Cl. 435/1.1; 435/374; 435/369; 435/370; 435/371

(57) ABSTRACT

The invention is the modification of organs, tissues and cells with a storage/reperfusion solution comprising siRNAs specific for genes whose expression is associated with loss of viability or cell damage. The presence of siRNAs in the storage/reperfusion solution minimizes and/or prevents organ, tissue and cell damage such that the organs, tissues or cells can be used for in vivo transplantation. The invention is also directed generally to methods for maintaining organs, tissues and cells in a viable state using the storage/reperfusion solution.

### FIELD OF THE INVENTION

The invention is directed to the preservation of organs, tissues and cells during storage, reperfusion and transport. More particularly, the invention is directed to the modification of organs, tissues and cells with a storage/reperfusion solution that minimizes and/or prevents organ, tissue and cell damage such that the organ, tissue or cells can be used for in vivo transplantation. The invention is also directed generally to methods for maintaining organs, tissues and cells in a viable state.

### BACKGROUND OF THE INVENTION

Dendritic cells (DC) are key controllers of immune function as they are capable of inducing both immune stimulation and immune suppression. A key factor deciding whether a DC will be stimulatory or inhibitory is expression of soluble and membrane-bound signals. For example, inhibition of CD80, CD86, and IL-12 on DC endows them with the ability to suppress immune system activation. On the other hand, suppression of DC-inhibitory signals such as PD-1L allows the DC to become a more potent activator of T cell responses. Manipulation of such DC-derived signals has been performed by antibody blockade (Goldberg et al., Transpl Int, 1994. 7 Suppl 1: p. S252-4], antisense oligonucleotides [Liang et al., Transplant Proc, 2001. 33(1-2): p. 235)], and chemical immunosuppressants [Lagaraine, et al., 2003. 75 (9Supplm): p. 37S-42S). Unfortunately, all of these approaches possess significant drawbacks that limit their entrance into the clinic.

Recently, small interfering RNA (siRNA) has been developed, which provides a more potent, specific and long-lasting method of gene suppression compared to other known gene suppression methods (Scherr et al., Curr Med Chem, 2003. 10(3): p. 245-56). It has been demonstrated that low concentrations of siRNA may be used to silence DC cytokine production, as well as modulate the ability of DC to activate T cells. The Applicant's PCT CA03/00867 describes the manipulation of immunological cells, including DC, through the use of siRNA, as well as methods of modifying T cell responses using siRNA-silenced DC.

Transplantation of tissues and organs requires a supply of viable tissues and organs. Autologous and heterologous transplantation is limited by the time a tissue or organ can be maintained viable prior to the transplantation of the tissue or organ. Donor organs are subjected to flushing and storage in hypothermic conditions (4° C.) in specially formulated perfusion solutions in order to wash out debris and to decrease damage during transportation. Such solutions can be broadly differentiated as “extracellular” and “intracellular” based on the physiological milieu they mimic. The solutions contain active components which minimize cell swelling caused by hypothermia, inhibit acidosis due to ischemia, minimize extracellular expansion, minimize free-radical damage and supply a substrate for regenerating ATP during reperfusion. U.S. Pat. No. 5,110,722 and U.S. Patent Application Serial Nos. 2003/0109433 and 2005/0100876 describe organ storage solutions to attain better graft function. Organ storage solutions have also been modified by the addition of free-radical protectors, caspase inhibitors and angiotensin converting enzyme inhibitors (Baker et al., J Surg Res, 1999. 86(1): p. 145-9; McAnulty et al., Cryobiology, 1996, 33(2): P. 217-25; Natori et al., Liver Transpl, 2003. 9(3): p. 278-84; Randsbaek et al., Scand Cardiovasc J, 2000 192(1): p. 31-40).

Immune modulation has also been attempted by the use of a perfusion solution that inhibits donor co-stimulatory molecules and enhances graft survival (Wekerle et al., Curr Opin Immunol, 2002. 14(5): p. 592-600). Perfusion of kidney grafts with naked antisense DNA has been demonstrated to suppress the expression of intracellular adhesion molecule-1 (Chen et al., Transplantation, 1999. 68(6): p. 880-7). Similarly, inhibition of NF-KB activity in cardiac allografts was performed by the addition of decoy oligonucleotides to an organ storage solution (Vos et al., Faseb J, 2000. 14(5): p. 592-600). However, both of these approaches require high concentrations of oligonucleotides for induction, resulting in marginal efficacy.

U.S. Patent Application Publication No. 2006/0073127 describes the preparation of tissues for transplantation using a selected RNAi agent. However, the methods use short perfusion times and tissue storage was maintained at typical cold storage temperatures of 4° C. Further, the use of naked siRNA was stated to result in poor diffusion in the tissue and thus was ineffective. Bradley et al., (Transplantation Proceedings, 37, 233-236, 2005) simply demonstrate the use of siRNA for imaging of pancreatic islet cells.

It is therefore highly desirable to provide an improved organ storage/reperfusion solution and methods of use thereof, whereby organs, tissues and cells are effectively modified to decrease immune recognition thus leading to improved viability and recipient acceptance.

### SUMMARY OF THE INVENTION

The invention is a composition for the preservation of the viability of organs, tissues and/or cells during ex vivo storage, reperfusion and transport such that the organs, tissues and/or cells may be successfully used for in vivo transplantation. The transplantation may be autologous or heterologous. The invention is also directed to methods of use of such compositions for maintaining organs, tissues and/or cells ex vivo in a viable state for transplantation. The invention is further directed to the modified organs, tissues and/or cells per se. The compositions of the invention comprise siRNA that is specifically targeted/directed to the silencing of gene expression that is responsible or associated with a loss of viability and/or cell damage in organs, tissues and/or cells. In aspects of the invention the genes for targeting are those involved in apoptosis, immuno-inflammatory reactions and complement activation.

According to an aspect of the present invention is a composition for maintaining cells, tissues and/or organs in a viable state. The cells, tissues and/or organs are maintained in a viable state ex vivo during storage and in vivo during reperfusion. The composition comprises one or more siRNA specific for a gene whose expression is associated with loss of viability or cell damage in ex vivo tissues or organs. As such, the siRNA targets the expression of such genes. Such genes may include one or more of apoptosis genes, immuno-inflammatory genes and complement genes and various combinations of such different genes.

According to an aspect of the present invention is a composition for maintaining cells, tissues and/or organs in a viable state ex vivo during storage and in vivo during reperfusion, the composition comprising siRNA specific for genes whose expression is associated with loss of viability or cell damage in ex vivo tissues or organs.

In aspects of the present invention, the composition is a storage solution that permits storage of cells, tissues and/or organs at room or refrigerated temperatures for periods of time longer than is possible using present clinically accepted solutions. In aspects of the invention, the composition is provided at about 37° C. and comprises combinations of siRNA targeting one or more apoptosis genes, one or more immuno-inflammatory genes and one or more complement genes and various combinations thereof.

According to the present invention, there is provided a composition for maintaining the viability of cells, tissues and/or organs during reperfusion and ex vivo such that the cells, tissues and/or organs are viable for transplantation, the composition comprising one or more siRNA targeting the expression of one or more apoptosis genes, immuno-inflammatory genes and complement genes. In aspects of the invention, the composition may further comprise other agents known to aid in the viability of cells, tissues and/or organs ex vivo as later herein described.

According to another aspect of the present invention is a method of storing or transporting cells, tissues and/or organs while maintaining them in a viable state prior to transplantation.

According to a further aspect of the present invention is a method of delaying the detrimental effects of ischemia on organ, tissue and/or cell viability, and to a storage solution suitable for use in such a method.

According to a further aspect of the present invention is a method of delaying the detrimental effects of apoptosis on organ, tissue and/or cell viability, and to a storage solution suitable for use in such a method.

According to a further aspect of the present invention is a method of delaying the detrimental effects of inflammation on organ, tissue and/or cell viability, and to a storage solution suitable for use in such a method.

According to yet a further aspect of the present invention is a method of maintaining the viability of cells, tissues and/or organs during reperfusion prior to excision from a mammalian host.

According to yet another aspect of the present invention is a method for altering cells, tissues and/or organs resulting in the viability of the cells, tissues and/or organs during storage and reperfusion prior to and during transplantation.

According to another aspect of the invention is a method for maintaining the viability of a tissue or an organ maintained ex vivo prior to transplantation, comprising contacting the tissue or organ with at least one siRNA specific for a gene whose expression is associated with loss of viability or cell damage in ex vivo tissues or organs. Such genes may include one or more of apoptosis genes, immuno-inflammatory genes, complement genes and combinations thereof.

According to another aspect of the invention is a method for protecting a tissue or an organ of a mammal against ischemic and/or reperfusion injury comprising contacting the tissue or organ with at least one siRNA specific for a gene whose expression is associated with ischemic and/or reperfusion injury. In aspects, the siRNA is provided as a composition. In further aspects, the siRNA composition is provided at temperatures of over 4° C., in still further aspects at temperatures about 37° C.

According to an aspect of the invention is a method for maintaining the viability of a tissue, cells or an organ maintained ex vivo prior to transplantation, the method comprising contacting the tissue, cells or organ with at least one siRNA specific for a gene whose expression is associated with loss of viability or cell damage in ex vivo tissues, cells or organs, wherein said contact is made at temperatures of over about 4° C.

According to another aspect of the invention is a method for protecting a tissue, cells or an organ of a mammal against ischemic and/or reperfusion injury comprising contacting the tissue, cells or organ with at least one siRNA specific for a gene whose expression is associated with ischemic and/or reperfusion injury.

According to a further aspect of the present invention is a method of storage of a cell, tissue or organ in a viable state, the method comprising:

i) contacting a cell, tissue or organ to be stored with a solution comprising siRNA; and

ii) maintaining the cell, tissue or organ in contact with the solution at a sub-ambient temperature in a non-frozen state.

According to a further aspect of the present invention is a method of storage of a cell, tissue or organ in a viable state, the method comprising:

i) contacting a cell, tissue or organ to be stored with a solution comprising siRNA; and

ii) maintaining the cell, tissue or organ in contact with the solution at a temperature of about 37° C.

According to yet another aspect of the present invention is an altered cell, tissue or organ, wherein said altered cell, tissue or organ comprises siRNA targeted to silence the expression of one or more apoptosis genes, immuno-inflammatory genes and complement genes. In aspects, the cell, tissue and/or organ is provided and maintained ex vivo. In other aspects, the cell tissue and/or organ is provided in vivo, i.e. transplanted into a suitable mammalian recipient.

According to yet another aspect of the present invention is a method for preparing a tissue for transplantation, the method comprising:
exposing said tissue to a composition comprising siRNA targeted to silence the expression of one or more apoptosis genes, immuno-inflammatory genes and complement genes,
maintaining said exposure for a time sufficient to down-regulate said one or more apoptosis genes, immuno-inflammatory genes and complement genes.

In aspects said exposure is conducted at temperatures of about 4° C. to about 37° C. In further aspects, the tissue is a kidney.

According to another aspect of the invention is a method of storage of a cell, tissue or organ in a viable state, the method comprising:

i) contacting a cell, tissue or organ to be stored with a composition comprising at least one siRNA specific for a gene whose expression is associated with loss of viability or cell damage; and

ii) maintaining the cell, tissue or organ in contact with the solution at temperatures of from about sub-ambient up to about 37° C.

Further aspects and advantages of the present invention will be clear from a reading of the description that follows.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from the detailed description.

### BRIEF DESCRIPTION OF THE DRAWINGS

A preferred embodiment of the present invention will now be described more fully with reference to the accompanying drawings:

FIG. 1 shows the increased expression of the RelB gene in kidney ischemia after reperfusion. CD1 mice were made to experience an ischemic injury of the kidney using a warm ischemia/reperfusion model. The renal vein and artery of the left kidney were clipped for 25 minutes at 37° C. The right kidney was then removed. The RNA was extracted from the kidney after the clipping experiments after indicated time points of clipping. Controls are the RNA from normal mice. The expression of RelB was determined by RT-PCR.

FIG. 2 shows the increased expression of the Fas gene in kidney ischemia after reperfusion. CD1 mice were made to experience an ischemic injury of the kidney using a warm ischemia/reperfusion model. The renal vein and artery of the left kidney were clipped for 25 minutes at 37° C. The right kidney was then removed. The RNA was extracted from the kidney after the clipping experiments after indicated time points of clipping. Controls are the RNA from normal mice. The expression of Fas was determined by RT-PCR.

FIG. 3 shows the increased expression of the caspase 8 gene in kidney ischemia after reperfusion. CD1 mice were made to experience an ischemic injury of the kidney using a warm ischemia/reperfusion model. The renal vein and artery of left kidney were clipped for 25 minutes at 37° C. The right kidney was then removed. The RNA was extracted from the kidney after the clipping experiments after the indicated time points of clipping. Controls are the RNA from normal mice. The expression of Caspase 8 was determined by RT-PCR.

FIG. 4 shows the increased expression of the caspase 3 gene in kidney ischemia after reperfusion. CD1 mice were made to experience an ischemic injury of the kidney using a warm ischemia/reperfusion model. The renal vein and artery of left kidney were clipped for 25 minutes at 37° C. The right kidney was then removed. The RNA was extracted from the kidney after clipping experiments after indicated time points of clipping. Controls are the RNA from normal mice. The expression of Caspase 3 was determined by RT-PCR.

FIG. 5 shows the increased expression of the C3 gene in kidney ischemia after reperfusion. CD1 mice were made to experience an ischemic injury of the kidney using a warm ischemia/reperfusion model. The renal vein and artery of left kidney were clipped for 25 minutes at 37° C. The right kidney was then removed. The RNA was extracted from the kidney after clipping experiments after indicated time points of clipping. Controls are the RNA from normal mice. The expression of C3 was determined by RT-PCR.

FIG. 6 shows the increased expression of the C5aR gene in kidney ischemia after reperfusion. CD1 mice were made to experience an ischemic injury of the kidney using a warm ischemia/reperfusion model. The renal vein and artery of left kidney were clipped for 25 minutes at 37° C. The right kidney was then removed. The RNA was extracted from the kidney after clipping experiments after indicated time points of clipping. Controls are the RNA from normal mice. The expression of C5aR was determined by RT-PCR.

FIG. 7 shows the silencing of the caspase-3 gene using siRNA in vitro. Caspase 3-siRNA-expression vectors were transfected into a macrophage cell line that expresses Caspase 3. 48-hours after gene silencing, the total RNA was extracted and gene expression was determined by RT-PCR.

FIG. 8 shows the silencing of caspase-8 gene using siRNA in vitro. Caspase 8-siRNA-expression vectors were transfected in to a macrophage cell line that expresses Caspase 8. 48-hours after gene silencing, the total RNA was extracted and the gene expression was determined by RT-PCR.

FIG. 9 shows the silencing of the RelB gene using siRNA in vitro. RelB cDNA and RelB-siRNA were co-transfected into a macrophage cell line. 48-hours after gene transfection and silencing, the total RNA was extracted and the gene expression was determined by RT-PCR. Both synthesized siRNA and siRNA-expression vectors are demonstrated to potently silence enhanced expression of RelB.

FIG. 10 shows the silencing of the Fas gene using siRNA in vitro. Fas-siRNA-expression vectors were transfected into a macrophage cell line that expresses Fas. 48-hours after gene silencing, the protein was extracted and the Fas expression was determined by Western blotting.

FIG. 11 shows the silencing of the caspase 3 Gene in the kidney. CD1 mice were i.v. injected with 50 μg of siRNA specific to caspase 3 gene. The renal vein and artery were clipped for 25 minutes at 37° C. 48 hours after gene silencing, the total RNA was extracted from the kidney and the Caspase expression was determined by RT-PCR.

FIG. 12 shows the silencing Fas gene in kidney. CD1 mice were i.v. injected with 50 μg of siRNA specific to Fas gene. The renal vein and artery were clipped for 25 minutes at 37° C. 48 hours after gene silencing, the total RNA was extracted from kidney and the Fas expression was determined by RT-PCR.

FIGS. 13A and 13B demonstrate the prevention of ischemia reperfusion injury in the kidney by the silencing of immunoinflammatory genes. CD1 mice were i.v. injected with 50 μg of siRNA specific to TNFα and RelB genes alone or in combination. After gene silencing, renal vein and artery were clipped for 25 minutes at 37° C. The renal function was determined by detecting blood creatinine (A) and BUN (B) levels 24 hrs after reperfusion.

FIGS. 14A and 14B demonstrate the prevention of ischemia reperfusion injury in the kidney by the silencing of apoptotic genes. CD1 mice were i.v. injected with 50 μg of siRNA specific to Caspase 3, Caspase 8 and Fas genes alone or in combination. After gene silencing, renal vein and artery were clipped for 25 minutes at 37° C. The renal function was determined by detecting blood creatinine (A) and BUN (B) levels 24 hrs after reperfusion.

FIGS. 15A and 15B demonstrate the prevention of ischemia reperfusion injury in the kidney by combinational gene silencing. CD1 mice were i.v. injected with 50 μg of siRNA specific to: group 1, caspase 3, caspase 8 and Fas genes; group 2, TNFα and RelB genes; group 3, C3 and C5aR genes, and group 4, all above genes (S-mix). After gene silencing, renal vein and artery were clipped for 25 minutes at 37° C. The renal function was determined by detecting blood creatinine (A) and BUN (B) levels were determined 24 hrs after reperfusion.

FIG. 16 demonstrates that gene silencing prevents the death of the mice after ischemic reperfusion injury. CD1 mice were i.v. injected with 50 μg of a mixture of siRNA mixture specific to Caspase 3, Caspase 8, Fas, C3, C5aR, TNFα and RelB genes. After gene silencing, renal vein and artery were clipped for 35 minutes at 37° C.

FIGS. 17A-D demonstrates In vitro gene silencing of the C3 and Caspase 3 genes. (C3—17A & 17B) L929 cell lines were transfected with C3 cDNA vectors using lipofectamine 2000, and co-transfected with C3 siRNA, empty vectors or non-siRNA (control). 24 hrs after transfection, cells were harvested to extract total RNA. Transcripts of C3 and GAPDH were determined using RT-PCR (17A) and quantitative PCR (17B). (Caspase 3—17C & 17D) For silencing the Caspase 3 gene L929 cells were transfected with Caspase 3 siRNA, empty vectors, or non-siRNA (control). 24 hrs after transfection, expression of Caspase 3 was detected using RT-PCR (17C) and quantitative PCR (17D).

FIGS. 18A-B demonstrate the silencing of C3 in vivo. (18A) Upregulated expression of C3 and Caspase 3 genes in the kidney after I/R injury. Left kidney was subjected to clamping for 25 min as described in the examples section. Kidneys were harvested at indicated time points after clamping. The expression of C3 and Caspase 3 was detected by RT-PCR. (18B) Mice were pretreated with 50 μg of C3 siRNA and Caspase 3 siRNA, or empty vectors for 48 hrs followed by I/R experiments. Kidneys were harvested, 24 hrs after I/R, for determination of C3 and Caspase 3 gene expression using RT-PCR.

FIGS. 19A-D show histological changes in I/R injury kidneys. Mice were treated with siRNA and I/R injury experiments were performed, as described in FIG. 18B. 24 hrs after I/R, kidney tissues were harvested and sectioned, then stained with H&E. (19A) normal unclamped kidney; (19B) PBS-treated I/R kidney; (19C) empty vectors-treated I/R kidney; (19D) C3 siRNA and Caspase 3 siRNA-treated I/R kidney.

FIG. 20 shows siRNA protects kidneys from I/R injury using Caspase 3 and C3. a) Mice were treated with 50 mg siRNA i.v. or empty vectors 48 hrs prior to I/R injury experiment. 24 hrs after I/R, blood was collected to determine levels of BUN and serum creatinine. Data shown are means ±SEM. p values were compared with PBS-treated control groups using Student's t test. Mice were treated with 50 mg siRNA i.v. or empty vectors 48 hrs prior to I/R injury experiment. The survival of mice was observed by the eighth day after I/R injury.

FIGS. 21A-B show the increased expression of caspase-3/8 in kidney IRI. (21A) Caspase-3 expression detected by RT-PCR. Left kidney was subjected to clamping for 25 min as described in Materials and Methods. 24 hours after clamping, kidney was harvested and total RNA was extracted. Transcriptions were amplified using primers specific to caspase-3 (21A) and caspase-8 (21B), and GAPDH genes. Data shown represent experiments performed on five animals per group.

FIGS. 22A-D show the silencing of caspase-3/8 in vivo. 50 μg of pRNAT U6.1 vectors that contains caspase-3 siRNA or caspase-8 siRNA. Controls included a blank vector (non-siRNA) treatment and PBS-treatment groups. 48 hours after gene silencing, kidneys were clamped for 25 min. 24 hours after clamping, kidney tissues were harvested and total RNAs were extracted. Transcripts of caspase-3 (22A & 22C) and caspase-8 (22B & 22D), and GAPDH were determined by RT-PCR (22A & 22B) as well as quantitative real-time PCR (22C & 22D).

FIGS. 23A-B show that siRNA protects renal function in IRI. Renal pedicles were clamped for 25 min. Blood was collected before clamping (0 h) and 24 h after reperfusion (24 h) to determine levels of BUN (23A) and serum creatinine (23B). Data shown are mean±SEM (*p<0.05, caspase-3/8-siRNA treated versus untreated and clamped mice).

FIG. 24 shows that siRNA protects against lethal kidney ischemia. Mice were i.v. treated with 50 μg caspase-3 and 8-siRNA vectors, or blank vectors, or PBS, following clamping for 35 min. Survival of siRNA treated (n=10), and control vector treated (n=10) or PBS control mice was observed over 8 days. (p<0.05, caspase-3/8 siRNA treated versus untreated and clamped mice).

FIG. 25 shows RelB siRNA silences the RelB gene expression in vitro. Silencing of RelB mRNA analyzed by RT-PCR of RelB and GAPDH expression from L929 cell lines treated with RPMI, transfection reagent, empty vector, scramble siRNA and RelB siRNA. A representative sample is shown.

FIGS. 26A-B shows RelB expression was upregulated after ischemia reperfusion in vivo. RelB silence efficacy was tested by RT-PCR of RelB and GAPDH expression in kidney tissue homogenates from untreated mice or mice clipped 25 min at 24 hours time point (26A,B). Downregulation of RelB gene expression by siRNA. Animals were treated as described in the example section. The tissues were from untreated animals, treated with RelB siRNA for 24 hours and 48 hours. RelB gene expression was assessed by RT-PCR as described in Methods. The results also expressed as mean +/−SD of fold change compare with GAPDH.

FIGS. 27A-B show the improvement of renal function after RelB siRNA silencing. Mice received a single hydrodynamic injection of siRNA in PBS (filled bars) or just PBS (dotted) 2 days before, as described in example section. Samples were harvested 24 hours after clamping, as indicated. (27A) BUN increased in the positive control animals (PBS), but decreased in the siRNA treated mice. (27B) Creatinine level were higher in the mice injected with PBS, But not in the in the group treated with siRNA.

FIGS. 28A-B shows the protection of siRNA on kidney ischemia injury. Mouse kidneys were subjected to 25 minutes of ischemia followed by 24 hours of reperfusion. Kidney tissues were fixed in 10% neutral-buffered formalin for 48 hours and then embedded in paraffin. (28A) Ischemia reperfusion caused severe neutrophile infiltration tubli vaculisation and cast formation. (28B) RelB siRNA reduced the damage of ischemia reperfusion.

FIG. 29 shows the hydrodynamic injection of RelB siRNA protects mice from lethal kidney ischemia reperfusion injury: survival after 35 minutes of kidney ischemia and perfusion.

FIG. 30 shows that hearts harvested from mice and preserved in siRNA composition of the invention comprising siRNAs for RelB, Fas, caspase-3, caspase-8, TNFα, C5aR and C3 could be transplanted into a recipient mouse and the hearts survived compared to the control hearts that died. The hearts were harvested from BALB/c mice, preserved in a siRNA composition of the invention containing UW solution for 48 hours at 4° C. In vitro preserved organs were used for heart transplantation as donor. The recipients were syngeneic strain BALB/c mice. Hear beats are monitored daily. The controls were preserved in UW solution only. The control organs were dead after 48 hours preservation using UW solution. siRNA treated hearts beat until the end point of experiment (40 days). The pictures shown the hearts organs 40 days after transplantation.

### DETAILED DESCRIPTION OF THE PREFERRED EMBODIMENTS

The invention is directed to a composition that can maintain the viability of cells, tissues and/or organs ex vivo such that they can be stored, transported and then used for transplantation in vivo. The composition comprises one or more siRNA that are specifically directed to target a gene selected from the group consisting of apoptosis genes, immuno-inflammatory genes, complement genes and combinations thereof. The targeted siRNA effectively inhibits (down-regulates) the targeted gene expression in the cells, tissues and organs leading to the viability of the cells, tissues and organs. The invention also provides methods of using such compositions. Advantageously, siRNA is a non-viral method of altering gene expression and thus would be preferred for immunosuppressed transplant patients.

By ‘viability’ or ‘viable’ it is meant that the cells, tissues or organs remain capable of primary function. As such, the cells, tissues or organs can be perfused, stored, transported and then transplanted in vivo to a recipient in such a manner that they substantially retain their function.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference. In aspects, an siRNA comprises between about 15-30 nucleotides or nucleotide analogs, in other aspects between about 16-25 nucleotides (or nucleotide analogs), and in further aspects between about 18-23 nucleotides (or nucleotide analogs), and yet in further aspects between about 19-22 nucleotides (or nucleotide analogs) (e.g., 19, 20, 21 or 22 nucleotides or nucleotide analogs).

As used herein, the term “antisense strand” of an siRNA or RNAi agent refers to a strand that is substantially complementary to a section of about 10-50 nucleotides, e.g., about 15-30, 16-25, 18-23 or 19-22 nucleotides of the mRNA of the gene targeted for silencing. The antisense strand has sequence sufficiently complementary to the desired target mRNA sequence to direct target-specific RNA interference (RNAi), e.g., complementarity sufficient to trigger the destruction of the desired target mRNA by the RNAi machinery or process. The term “sense strand” of an siRNA or RNAi agent refers to a strand that is complementary to the antisense strand. Antisense and sense strands can also be referred to as first or second strands, the first or second strand having complementarity to the target sequence and the respective second or first strand having complementarity to said first or second strand. A guide strand then refers to a strand of an RNAi agent, e.g., an antisense strand of an siRNA duplex, that enters into the RISC complex and directs cleavage of the target mRNA. The term “guide strand” is often used interchangeably with the term “antisense strand” in the art.

A “target gene” is a gene whose expression is to be selectively inhibited or “silenced.” This silencing is achieved by cleaving the mRNA of the target gene by an RNAi pathway or process. In the present invention a target gene is a gene whose expression is associated with loss of viability or cell damage. Such genes are selected from one or more apoptosis genes, one or more complement genes, one or more immuno-inflammatory genes and combinations thereof.

The term “perfusion”, as used herein, refers to the act of pouring over or through, especially the passage of a fluid through the vessels of a specific organ. In specific embodiments of the instant invention, fluids containing siRNA are perfused through the vasculature of transplant tissues.

The terms “apoptosis” or “programmed cell death,” refers to the physiological process by which unwanted or useless cells are eliminated during development and other normal biological processes. Apoptosis, is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy. Apoptosis may also be triggered by external events and stimuli, such as ischemic injury in the case of certain preferred embodiments of the instant invention. Cells undergoing apoptosis show characteristic morphological and biochemical features.

“Inhibition of gene expression” refers to the absence (or observable decrease) in the level of protein and/or mRNA product from a target gene. “Specificity” refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

It is asserted that any type of cell, tissue or organ that is desired and used for transplantation may be used in the present invention as is understood by one of skill in the art. Representative examples of organs are but not limited to heart, liver, lung, pancreas and kidney. Cells (i.e. pancreatic islet cells, or other tissues (i.e. blood vessels) are also encompassed by the present invention.

Briefly, siRNA is a method of RNA interference used to inhibit gene expression in mammalian cells (Bertrand et al., 2002. Biochem Biophys Res Commun 296(4):1000-1004). SiRNA are short double stranded RNA molecules of approximately 21-25 base pair length which interact with cytoplasmic proteins to form the intracellular RNAi induced silencing complex (RISC). RISC uses the antisense strand of the siRNA to bind and cleave the associated mRNA sequence, which is subsequently degraded by non-specific Rnases. SiRNAs function in the cytoplasm and require lower concentrations to achieve target gene knockdown as compared to antisense oligonucleotides.

When organs are harvested for transplantation, the ensuing period of hypoxia, followed by reperfusion of the organ, is accompanied by substantial tissue damage, including endothelial cell apoptosis and parenchymal dysfunction. Such ischemia/reperfusion (I/R) injury can involve inflammatory reactions controlled in part by transcription factor NF-κB and its subunit Rel B, activation of apoptotic pathways, for example by caspases, activation of the complement system, and activation of immuno-inflammatory genes such as Fas and TNF-alpha genes.

The present invention is based on the use of siRNA to target the expression of those genes that may lead to the non-viability and reduced viability of the cells, tissues and/or organs for transplantation. Such genes may include for example apoptosis genes, immuno-inflammatory genes and complement genes and combinations thereof. The invention provides compositions and methods using such compositions that comprise one or more apoptosis genes, one or more immuno-inflammatory genes, one or more complement genes and combinations thereof. In this manner a more effective approach is provided that is more effective to maintain tissues/cells in a viable state leading to improved transplantation success.

In aspects of the invention, suitable apoptosis genes may include but not be limited to caspase3 gene, caspase8 gene and the fas gene. Suitable immuno-inflammatory genes may include but not be limited to TNF-alpha gene and the RelB gene. Suitable complement genes may include but not be limited to C3 and C5aR. It is also understood by one of skill in the art that combinations of genes may be targeted using siRNA as is desired. Those of skill in the art would readily understand what other genes may be used in the compositions of the present invention which are either involved in apoptosis, immuno-inflammation and complement genes which are involved in the initiation of coagulation. Those of skill in the art would also readily understand the short sequences of the genes that can be used in the present invention, a few of which are listed herein in the example section as representative examples only and are not meant to be limiting. Furthermore, representative examples of apoptosis genes for use in the present invention is shown in Table One and representative example of complement genes for use in the present invention is shown in Table Two. One of skill in the art may gain the sequences of such genes to use for the siRNA in the compositions and methods of the present invention.

The present invention now demonstrates in one embodiment, using an accepted model of ischemia/reperfusion (I/R) injury system in mice, that perfusion of an organ undergoing I/R injury with siRNA specifically targeted to one or more of the above-described genes associated with tissue-damaging reactions reduces expression of these genes, thus preventing the conditions for tissue damage and improves the viability of the organ. In this embodiment of the invention, the maintenance of good renal function in siRNA-treated kidneys has been demonstrated in spite of an I/R insult which produces renal damage in control organs. This illustrates the improved viability provided by the methods and compositions of the invention. The suppression of gene expression was seen when the organ undergoing ischemia and reperfusion was maintained at 37° C., i.e. much more rigorous conditions than those normally employed for transplantation organs, that are normally maintained at a low temperature.

The present invention can be practiced on cells, tissues or organs as is understood by one of skill in the art. Suitable organs may be but are not limited to heart, liver, lung, pancreas and kidney. In aspects of the invention the delivery of siRNA is during or through the procurement of an organ (e.g., via administration via the vasculature of said organ) and during or through isolation of transplantable cells from the organ. The siRNA composition and method may be used in several instances during the organ procurement process: 1) pre-procurement via intravenous perfusion in the deceased donor; 2) via organ perfusion prior to packaging for transport; and/or 3) in cell culture

More specifically, tissues or organs may be protected against cell damage by treatment with a selected siRNA either before being harvested for transplantation or after harvesting. For example, the tissue or organ can be reperfused using the siRNA composition of the invention for a desired time; the tissue or organ can be simply bathed and stored in the siRNA composition of the invention for a desired time; or the tissue or organ can be both reperfused and bathed and stored in the siRNA composition of the invention. Of course, this also applies to cells as is understood by those of skill in the art. In embodiments of the invention, the tissues or organs are perfused with a siRNA solution before harvesting. The perfusion of the desired organ for example may be up to about 30 or 60 minutes as is understood by one of skill in the art. The harvested perfused tissues or organs are then stored in the siRNA solution and this storage may be about up to 24 hours or 48 hours depending on the type of tissue or organ as is understood by one of skill in the art. As such the siRNA of the invention can be used in a variety of methods to prevent apoptosis of cells, to prolong the life of a tissue and organ, to prevent and/or minimize ischemic damage and to help prevent tissue/organ rejection because the tissue/organ is in a viable condition thus providing less detrimental autoimmune reaction. The composition of the invention can be used at a variety of temperatures including refrigerated temperatures of about 2-4° C. to body temperatures of about 37° C. as is well understood by one of skill in the art. The composition of the invention can be used using standard protocols known for reperfusion and bathing and storage of tissues, cells and organs.

In particular aspects of the invention the compositions and methods are effective at temperatures of over 4° C. and up to about 37° C. or more and still effectively inhibits/prevents/minimizes ischemic damage to cells, tissues and organs. This is particularly advantageous in that there is no need to worry about keeping a tissue or organ cold for transplantation.

The siRNA composition of the invention is administered to the organ or tissue or cells by perfusion with and/or by bathing the ex vivo tissue, organ or cells in a suitable physiological solution containing the siRNA (Hamar, P., et al., Proc Natl Acad Sci 2004; 101:41). For example, a commercially available organ storage solution such as but not limited to Collins Solution, (UW)-solution, Histidine-Typtophan-Ketoglutarate (HTK) Solution, ViaSpan™ (intracellular) and Celsior solution (extracellular) may be used (Muhlbacher et al., 1999, Transplant Proc 31(5):2069-2070). Furthermore, other known additives may also be used in combination with the siRNA of the invention in the composition. For example such additives may include but not be limited to superoxide dismutase and other free radical scavengers (Baker et al., 1999, J Surg Res 86(1):145-149; McAnulty and Huang 1996, Cryobiology 33(2): 217-225; McLaren and Friend 2003, Transpl Int 16(10):701-708), lazaroids, anti-apoptosis agents (El-Gibaly et al., 2004, Hepatology 39(6):1553-1562; Natori et al., 2003, Liver Transpl 9(3):278-284), calcium channel blockers (Arnault et al., 2003, Transplantation 76(1):77-83), intercellular adhesion molecule-1 inhibitors (Stepkowski et al., 1998, Transplantation 66(6):699-707; Chen et al., 1999, Transplantation 68(6):880-887), pentoxifylline (Randsbaek et al., 2000, Scand Cardiovasc J 34(2):201-208) and combinations thereof.

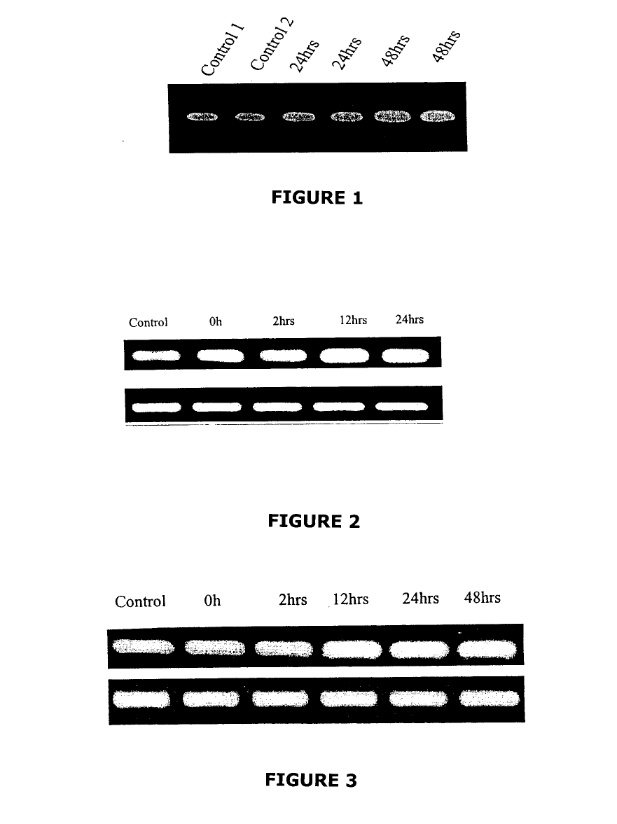

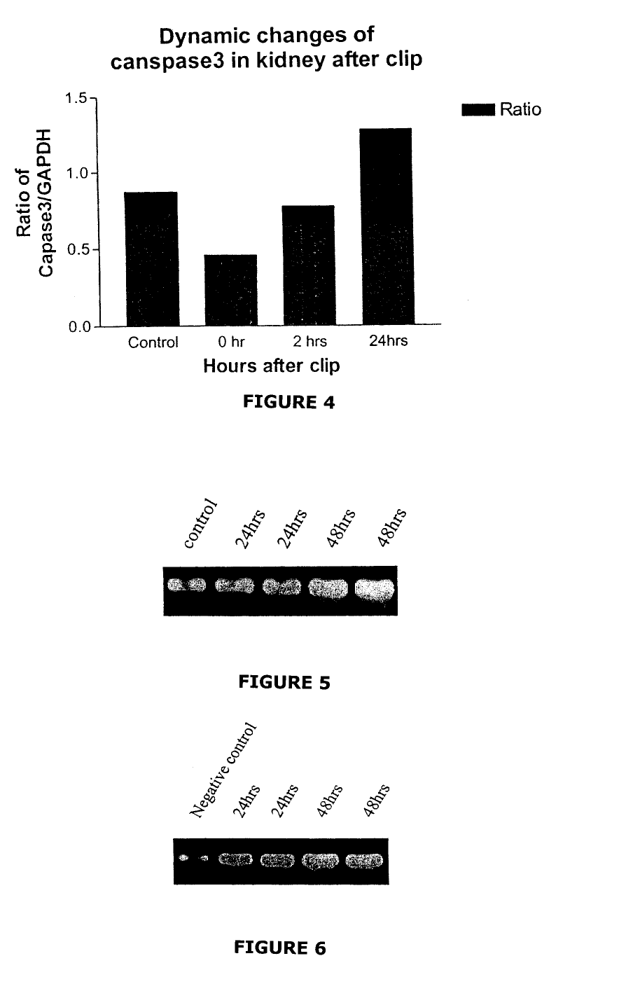

The siRNA may be used in a variety of strategies to silence a selected gene(s). For example, the following four strategies may be used: 1) Using a commercially pre-synthesized “siRNA pool” (Dharmacon Inc) consisting of 21 base-pair oligonucleotides that simultaneously target sites of a target gene such as for example 4 sites of the RelB gene (FIG. 1) which shows >75% gene silencing efficacy; 2) Using siRNA expression vectors (pSilencer™, Ambion Inc) with a pol III promoter that drives hairpin RNA expression to form a double-stranded RNA that serves as an endogenously expressed siRNA. Large amounts of Silencer-siRNA can be prepared through cloning techniques for in vitro (FIG. 2) and in vivo gene silencing; 3) Using siRNA-expression cassettes (SEC), which are generated as PCR products consisting of a hairpin siRNA template flanked by promoter and terminator sequences (FIG. 3). Once the SEC is transfected into cells, the hairpin siRNA is expressed from the PCR product and leads to gene silencing (FIG. 4). The advantage of SEC resides in the fact that it is extremely time efficient, which enables rapid screening for the most potent siRNAs amongst many candidate sequences; 4) Use SEC-vectors. Since SEC yield is small, the effective SEC subsequently must be cloned into viral or non-viral vectors (FIG. 5). Of course other strategies are encompassed by the present invention as is well understood by those of skill in the art.

Depending on the particular target gene or combination of genes and the dose of siRNA delivered, partial or complete loss of function for the target gene(s) is achieved. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or organism (as presented below in the examples) or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

The siRNA may be administered to the tissue or organ or cells in various forms, for example (1) as a naked siRNA oligonucleotide; (2) incorporated into an siRNA expression vector which drives hairpin RNA expression to form a double stranded RNA that serves as an endogenously expressed siRNA. For example, siRNA expression vectors may be constructed with pSilencer 2.0-U6 (Ambion Inc. Austin Tex.). The specific siRNA insert oligonucleotides should be designed according to user's instruction. The oligonucleotide contains 19-mer hairpin sequences specific to the mRNA target, a loop sequence separating the two complementary domains, two 3′-end overhang nucleotide and a poly thymidine tract to terminate transcription and 5′ single-stranded overhang for ligation into pSilencer with BamH1 and Hind III. Both sense and anti-sense hairpin siRNA-encoding oligonucleotides were annealed as an insert as described in Shi, Y., (2003), Trends Genet., v. 19, pp. 9-12; (3) as an siRNA expression cassette (SEC), generated as a PCR product consisting of a hairpin siRNA template flanked by promoter and terminator sequences, as described in Castanotto et al., (2002), Rna, v. 8, pp. 1454-60. Briefly, SECs were generated using a Silencer Express Kit (Ambion Inc, Austin Tex.). Sense and anti-sense hairpin siRNA template oligonucleotides for the precursor SEC were designed according to user's instruction. The oligonucleotides contain 19-mer hairpin sequences specific to the mRNA target, a loop separating the two complementary domains, two 3′-end overhang nucleotide. Briefly, two PCR reactions were performed to generate the precursor SEC using a Promoter Element (mouse U6) as template, a promoter PCR primer, and gene specific sense and anti-sense oligonucleotides. The first PCR product was used as template for the second PCR. The third PCR was performed to modify nucleotides at their 5′ ends and encode EcoR I and Hind III restriction sites (FIG. 1). Taq polymerase was used in PCRs (Invetregene Inc.); and (4) an SEC incorporated into a vector. Once effective SEC has been identified, the SEC was cloned into pVP22 with Mun I (compatible with EcoR I) and Hind III sites as described in Paul (2003), Mol. Ther., v. 7, pp. 237-247.

Any of the above forms of siRNA as described herein can be used and administered for mammalian use, including animals and humans. As for the amount of siRNA for use in the composition, about 1-50 micrograms per injection can be used in animals such as mice, which is sufficient to silence genes in vivo. About up to 0.2 to 100 μg/ml siRNA, per different siRNA, in solution may be used for flushing and storing heart and kidney organs. This includes any range therein between. A dosage regime may be used as is understood by one of skill in the art. The dosage regime can be done over a period of minutes, hours or days and can use various dosages of siRNA. Of course, the amounts of siRNA used in the composition may vary depending on the particular type of tissue or organ and the size thereof and can be readily determined by one of skill in the art. Therefore, the ranges provided herein are a guide and may in fact be greater.

The siRNA may be directly introduced into the cell (i.e., intracellularly), tissue, organ, allograft or organism; or introduced extracellularly into a cavity, interstitial space, into the circulation of an organism, introduced orally, or may be introduced by bathing a cell, tissue, organ, allograft or organism in a solution containing the siRNA. The bile or biliary system, vascular or extravascular circulation, the blood or lymph system, and the cerebrospinal fluid are sites where the siRNA may be introduced. In certain embodiments of the invention, the siRNA is provided to a transplanted tissue (e.g. an organ) by perfusion.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

TABLE ONE
Examples of Apoptosis Genes
Gene Families:

| Approved Stem Symbol | Approved Gene Family Name |
|---|---|
| BAG# | BCL2-associated athanogene # |
| BCL2L# | BCL2-like # Family specific webpage |
| BNIP# | BCL2/adenovirus E1B 19 kD-interacting protein # |
| CASP# | caspase #, apoptosis-related cysteine protease |
| TNFRSF# | tumor necrosis factor receptor superfamily, member |
| | # Family specific webpage |
| TNFSF# | tumor necrosis factor (ligand) superfamily, member # |
| TRAF# | TNF receptor-associated factor # |

Gene Symbols:

| Approved | Literature | | | Citation | Approved |
|---|---|---|---|---|---|
| Symbol | Aliases | Genbank ID | Location | PMID | Gene Name |
| AATK | AATYK, | AB014541 | 17q25.3 | 10083745; | apoptosis- |
| | KIAA0641 | | | 9734811 | associated |
| | | | | | tyrosine kinase |
| APAF1 | CED4 | NM_001160 | ? | 9267021 | apoptotic |
| | | | | | protease |
| | | | | | activating |
| | | | | | factor |
| BIRC2 | hiap-2 | L49431 | 11q22 | 8548810 | baculoviral IAP |
| | cIAP1 | | | | repeat- |
| | MIHB | | | | containing 2 |
| BIRC3 | cIAP2, | L49432 | 11q22 | 8548810 | baculoviral IAP |
| | hiap-1, | | | | repeat- |
| | MIHC | | | | containing 3 |
| BIRC4 | Xiap, hILP | U45880 | Xq25 | 8654366 | baculoviral IAP |
| | | | | | repeat- |
| | | | | | containing 4 |
| BIRC5 | — | U75285 | 17q25 | 8106347 | baculoviral IAP |
| | | | | | repeat- |
| | | | | | containing 5 |
| | | | | | (survivin) |
| BIRC6 | | AF265555 | 2p22-p21 | 10544019 | baculoviral IAP |
| | | | | | repeat- |
| | | | | | containing 6 |
| AP15 | AAC-11 | U83857 | ? | 9307294 | apoptosis |
| | | | | | inhibitor 5 |
| BAD | — | AF021792 | ? | 8929532 | BCL2- |
| | | | | | antagonist of |
| | | | | | cell death |
| BAK1 | CDN1 | U23765 | 6 | 7715730 | BCL2- |
| | | | | | antagonist/killer |
| | | | | | 1 |
| BAX | — | NM_004324 | 19q13.3-q13.4 | 8358790 | BCL2- |
| | | | | | associated X |
| | | | | | protein |
| BCL2 | — | M14745 | 18q21.3 | 2875799 | B-cell |
| | | | | | CLL/lymphoma2 |
| BCL10 | mE10 | AF082283 | 1p22 | 9989495 | B-cell |
| | CIPER | | | | CLL/lymphoma |
| | CARMEN | | | | 10 |
| BID | — | NM_001196 | 22q11.2 | 8918887 | BH3 interacting |
| | | | | | domain death |
| | | | | | agonist |
| BIK | NBK | U34584 | ? | 7478623 | BCL2- |
| | | | | | interacting |
| | | | | | killer |
| | | | | | (apoptosis- |
| | | | | | inducing) |
| BOK | — | AF027954 | ? | 9356461 | BCL2-related |
| | | | | | ovarian killer |
| CFLAR | CLARP CASH | AF005774 | 2q33-q34 | 9208847 | CASP8 and |
| | Casper | | | | FADD-like |
| | FLAME FLIP | | | | apoptosis |
| | I-FLICE | | | | regulator |
| | MRIT | | | | |
| CIDEA | CIDE-A | NM_001279 | ? | 9564035 | cell death- |
| | | | | | inducing |
| | | | | | DFFA-like |
| | | | | | effector a |
| CRADD | RAIDD | U84388 | 12q21.33- | 9044836 | CASP2 and |
| | | | q23.1 | | RIPK1 domain |
| | | | | | containing |
| | | | | | adaptor with |
| | | | | | death domain |
| DAD1 | — | D15057 | 14 | 8413235 | defender |
| | | | | | against cell |
| | | | | | death 1 |
| DAP | DAP1 | X76105 | 5p15.2 | 8530096 | death- |
| | | | | | associated |
| | | | | | protein |
| DAP3 | — | X83544 | 1q21 | 9284927; | death |
| | | | | 7499268 | associated |
| | | | | | protein 3 |
| DAPK1 | — | X76104 | 9q34.1 | 8530096 | death- |
| | | | | | associated |
| | | | | | protein kinase 1 |
| DAPK3 | — | AB007144 | 19p13.3 | 9488481 | death- |
| | | | | | associated |
| | | | | | protein kinase 3 |
| DAXX | DAP6 | AF00604 | 6p21.3 | 9215629 | death- |
| | | | | | associated |
| | | | | | protein 6 |
| EIF4G2 | DAP5 | X89713 | 11p13 | 9030685 | eukaryotic |
| | Nat1 | | | | translation |
| | | | | | initiation factor |
| | | | | | 4 gamma, 2 |
| HRK | DP5 | U76376 | ? | 9130713 | harakiri, BCL2- |
| | | | | | interacting |
| | | | | | protein |
| | | | | | (contains only |
| | | | | | BH3 domain) |
| FADD | MORT1 | U24231 | 11q13.3 | 7536190 | Fas |
| | | | | | (TNFRSF6)- |
| | | | | | associated via |
| | | | | | death domain |
| BIRC1 | — | U19251 | 5q13.1 | 7813013 | baculoviral IAP |
| | | | | | repeat- |
| | | | | | containing 1 |
| PDCD1 | — | L27440 | 2q37.3 | 7851902 | programmed |
| | | | | | cell death 1 |
| PDCD2 | — | NM_002598 | 6q27 | 7606924 | programmed |
| | | | | | cell death 2 |
| PDCD8 | AIF | AF100928 | Xq25-q26 | 9989411 | programmed |
| | | | | | cell death 8 |
| PDCD1 | — | | | | (apoptosis- |
| | | | | | inducing factor) |
| RIPK1 | RIP | U25994 | ? | 7538908 | receptor |
| | | | | | (TNFRSF)- |
| | | | | | interacting |
| | | | | | serine-threonine |
| | | | | | kinase 1 |
| RIPK2 | RICK, | AF078530 | 8q21 | 9575181 | receptor- |
| | RIP2 | | | | interacting |
| | CARDIAK | | | | serine-threonine |
| | | | | | kinase 2 |
| RNF7 | SAG | AF092878 | 3q22-q24 | 10230407; | ring finger |
| | ROC2 | | | 10082581 | protein 7 |
| STK17A | DRAK1 | AB011420 | 7p12-p14 | 9786912 | serine/threonine |
| | | | | | kinase 17a |
| | | | | | (apoptosis- |
| | | | | | inducing) |
| STK17B | DRAK2 | AB011421 | ? | 9786912 | serine/threonine |
| | | | | | kinase 17b |
| | | | | | (apoptosis- |
| | | | | | inducing) |
| TANK | I-TRAF | U59863 | 2q24-q31 | 8710854 | TRAF family |
| | | | | | member- |
| | | | | | associated |
| | | | | | NFKB activator |
| TRADD | — | L41690 | ? | 7758105 | TNFRSF1A- |
| | | | | | associated via |
| | | | | | death domain |

Genes without approved symbols:

| | | | | | Entrez |
|---|---|---|---|---|---|
| Literature | Proposed | Genbank | | Citation | Gene |
| Aliases | Symbol | ID | Location | PMID | ID |
| ALG-2 | PDCD6 | AF035606 | 5p15.2- | 8560270 | 10016 |
| | | | pter | | |
| Alix AIP1 | PDCD6IP | AJ005074 | ? | 10200558 | 10015 |
| BimL BOD | BCL2L11 | AF032458 | ? | 9430630 | 10018 |
| ES18, HES18 | PDCD7 | AF083930 | ? | 10037816 | 10081 |
| FLASH, CED-4 | CASP8AP2 | AF132726 | ? | ? | 9994 |
| TFAR19 | PDCD5 | AF014955 | ? | 9920759 | 9141 |
| TFAR15 | PDCD10 | AF022385 | ? | ? | 11235 |
| TOSO | CASP10AF1 | AF057557 | ? | 9586636 | 9214 |
| Nod1 CARD4 | APAF4 | AF126484 | 7p15-p14 | 10224040 | 10392 |
| DEDD DEFT | DEDD | AF043733, | ? | 9832420; | 9191 |
| FLDED1 | | AF083236, | | 9774341 | |
| | | AJ010973 | | | |
| MADD DENN | MADD | AB002356, | 11p11.21- | 8988362; | 8567 |
| KIAA0358 | | U44953, | p11.22 | 9796103 | |
| | | U77352 | | 9115275 | |
| DIO1, KIAA0333 | DATF1 | AB002331 | ? | 10393935 | 11083 |
| R1P3 | RIPK3 | AF156884 | ? | 10339433 | 11035 |
| FAF1, CGI-03 | FAF1 | AF132938 | 1 | 10462485 | 11124 |

TABLE TWO
Examples of Complement Genes

| | Gene | Chromosomal |
|---|---|---|
| Component (or subunit) | symbol | location |
| C1q: α chain | C1QA | 1p34.1-p36.3 |
| C1q: β chain | C1QB | 1p34.1-p36.3 |
| C1q: γ chain | C1QG | 1p34.1-p36.3 |
| C8: α chain | C8A | 1p32 |
| C8: β chain | C8B | 1p32 |
| C4 Binding Protein: α chain | C4BPA | 1q32 (a) |
| C4 Binding Protein: β chain | C4BPB | 1q32 (a) |
| Complement Receptor 1 (CD 35) | CR1 | 1q32 (a) |
| Complement Receptor 2 (CD 21) | CR2 | 1q32 (a) |
| Decay Accelerating Factor (CD 55) | DAF | 1q32 (a) |
| Membrane Cofactor Protein (CD 46) | MCP | 1q32 (a) |
| Factor H | HF | 1q32 (a) |
| Factor I | IF | 4q25 |
| C6 | C6 | 5p13 (b) |
| C7 | C7 | 5p13 (b) |
| C9 | C9 | 5p13 (b) |
| C2 | C2 | 6p21.3 (c) |
| Factor B | BF | 6p21.3 (c) |
| C4A (isotype) | C4A | 6p21.3 (c) |
| C4B (isotype) | C4B | 6p21.3 (c) |
| C8: γ chain | C8G | 9q22.3-q32 |
| C5 | C5 | 9q33 |
| Mannose Binding Lectin | MBL | 10q11.2-q21 |
| Perforin | PRF1 | 10q22 |
| Surfactant Proteins A1 and A2 | SFTPA1, A2 | 10q22-q23 |
| Surfactant Protein D | SFTPD | 10q22-q23 |
| Membrane Inhibitor of Reactive Lysis | CD59 | 11p13 |
| (MIRL, CD59) | | |
| C1 Inhibitor | C1NH | 11q11-q13.1 |
| C1r | C1R | 12p13 |
| C1s | C1S | 12p13 |
| Complement Receptor 3: α chain, =α-M | ITGAM | 16p11.2 |
| Integrin (CR3A, CD11A) | | |
| Vitronectin (S-protein) | VTN | 17q11 |
| C3 | C3 | 19p13.3-p13.2 |
| C5a receptor 1 | C5R1 | 19q13.3-q13.4 |
| Leucocyte Adhesion Molecule: β | ITGB2 | 21q22.3 |
| chain, =β-2 Integrin | | |
| (LCAMB, CD18) | | |
| Properdin | PFC | Xp11.4-p11.2 |

Footnotes:
(a) Regulators of complement activation (RCA) gene cluster
(b) Membrane attack complex (MAC) gene cluster
(c) MHC class III complement gene cluster

### EXAMPLES

Without intending to be limiting in scope, the following examples serve to illustrate various embodiments of the invention.

### General Methods Used

### Ischemia Protocol:

1) Mice (25-30 g) were anesthetized by intraperitoneal administration of Ketamine combined with inhalation of Enflurane.
2) Body temperature of the mice was kept constant by placing a warm pad (37° C.) beneath the animal.
3) Using a midline abdominal incision, the left renal pedicle was occluded for up to 35 minutes with a non-traumatic vascular clamp. After occlusion, 0.8 mL of prewarmed (37° C.) saline was placed in the abdominal cavity and the abdomen was covered with cotton soaked in sterile saline.
4) After removal of the clamp, the kidneys were observed for an additional 1 minute to see the color change indicative of blood reflow. Then the contralateral kidney was removed and the wound was closed in two layers. Control mice had identical surgical procedures except that vascular clamps were not applied.
5) Mice were sacrificed at 24 hours after reperfusion; blood samples were collected through inferior vena cava; and the left kidney was harvested for assessment of renal injury.
   siRNA Injection:
1) For systemic injection, synthetic siRNAs (in 0.8-1 ml PBS) was rapidly injected (within 10 sec) into one of the tail side veins or the penis vein. To dilate tail veins, the tail was immersed in warm water (50-55° C.), under ether narcosis for 5±1 sec.
2) For local injection, from a median laparotomy the left renal pedicle was visualized. Minimal preparation above the renal vessels was performed on the left side of the aorta to insert an occlusion clamp. The aorta and the vena cava were clipped, and the renal vein was punctured with a 30-gauge needle, to inject 0.1 ml of PBS containing siRNA. The needle was kept in place for 5 sec and than removed slowly, while applying compression to the renal vein for 30 sec with a little Avitene held with forceps. The Avitene was left in place thereafter. The aorta and vena cava clamp was removed immediately after the left renal pedicle was occluded for ischemia.
   siRNA Used in the Perfusion Studies to Silence Indicated Genes:

| | |
|---|---|
| Caspase3 gene: | GATCTATCTGGACAGTAGT |
| Caspase8 gene: | AAGCTCTTCTTCCCTCCCTAA |
| Fas gene: | AAGTGCAAGTGCAAACCAGAC |
| TNF-alpha gene: | AAGACAACCAACTAGTGGTGC |
| RelB gene: | GGA ATC GAG AGC AAA CGA A |
| C3 gene: | CTGTGCAAGACTTCCTAAAGA |
| C5aR gene: | GCACACTGTATGTGGTATTAA |

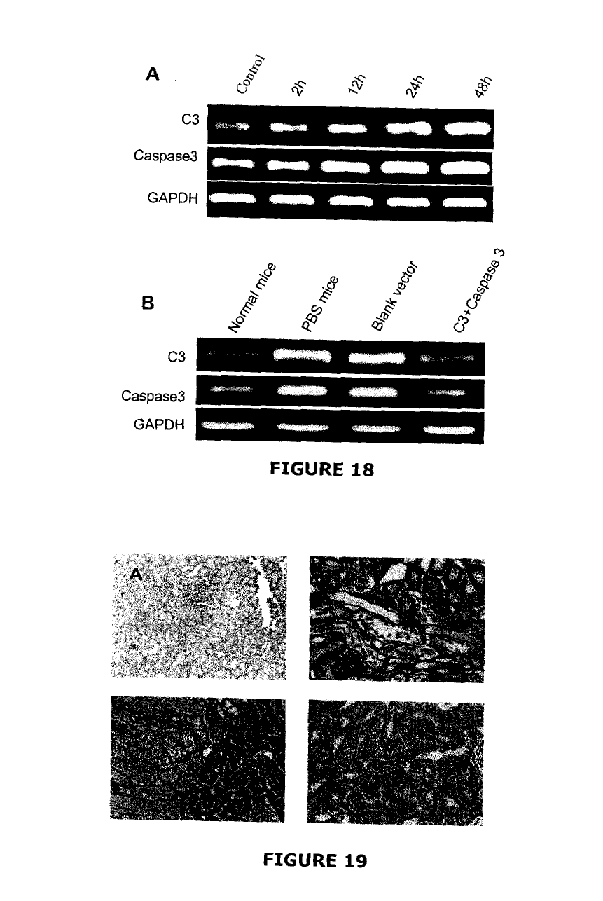

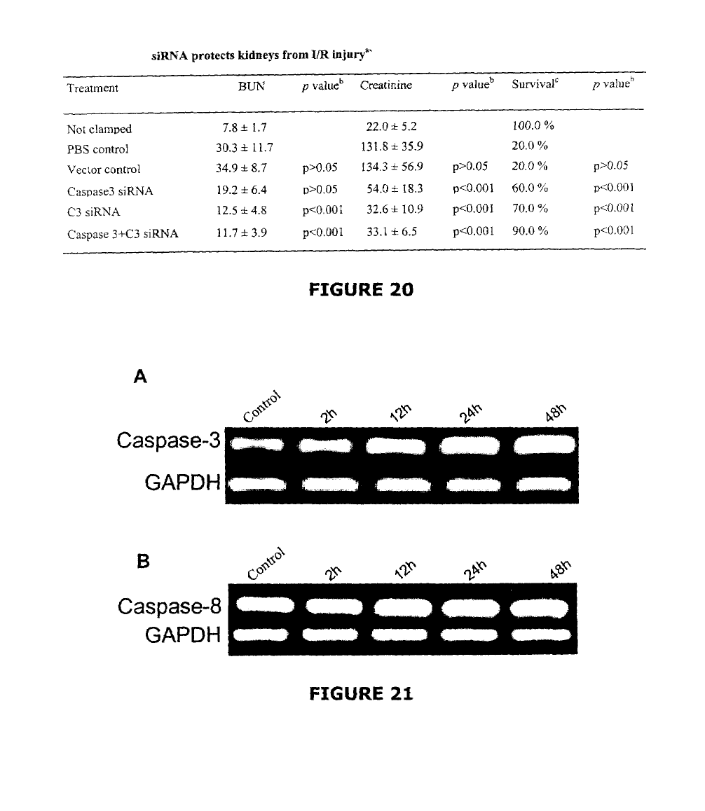

### General Methods Used (FIGS. 17-20)

C3 and Caspase 3 siRNA Design

The target sequences 5′-CTGTGCAAGACTTCCTAAAGA-3′ (specific to C3) and 5′-GGATCTATCTGGACAGTAGTT-3′ (specific to Caspase 3) were selected. The oligonucleotides containing sense and antisense of the target sequences and loop sequence, were synthesized, annealed, and constructed into a pRNAT-U6.1/Neo siRNA expression vector, which had a cGFP gene and a U6 promoter driving to express shRNA (Genescript, Piscataway, N.J.).

### In Vitro Silencing of the C3 and Caspase 3 Genes

L929 cells were transfected with C3 siRNA or Caspase 3 siRNA using lipofectamine 2000 (Invitrogen). The vehicle alone and scrambled (nonsense) siRNA were used as negative controls. Briefly, cells were plated into 12-well plates (2×10⁵ cells per well) and allowed to grow overnight, to reach 90% confluence. Cells were transfected with 2 μg of C3 siRNA, Caspase 3 siRNA or negative control siRNA plasmids in serum-reduced medium for 5 hrs, then incubated in complete medium for 24 hrs. RNA was extracted from the transfected cells 24 hrs after transfection.

### Renal I/R Injury Model

CD1 mice, 6-8 weeks old, were anesthetized with an intraperitoneal injection of ketamine (100 mg/kg) and xylazine (20 mg/kg) and placed on a heating pad to maintain their body temperature during surgery. Following abdominal incisions, renal pedicles were bluntly dissected and a microvascular clamp (Roboz Surgical Instrument, Washington, D.C.) was placed on the left renal pedicle for 25 min. During the procedure, animals were kept well hydrated with warm saline and at a constant temperature (37° C.). After 25 min of ischemia, the clamps were removed. The right kidney was resected.

### Assessment of Renal Function

Blood samples were obtained from the inferior vena cava 24 hrs after ischemia. Serum creatinine levels and blood urea nitrogen (BUN) were measured by the core laboratory at the London Health Sciences Centre in order to monitor renal function.

### Histology Detection

At 24 hrs post ischemia, kidneys were dissected from mice, and tissue slices were fixed in 10% formalin, and then processed for histology examination using standard techniques. Formalin tissue was embedded in paraffin and 5-μm sections were stained with H&E. These sections were examined in a blinded fashion by a pathologist. Histology changes in the cortex and medulla were examined.

Measurement of Renal C3 and Caspase 3 mRNA Levels by Reverse Transcriptase (RT)-PCR and Quantitative PCR

Total RNA was extracted from kidneys and cells using Trizol (Invitrogen). Total RNA was reverse-transcribed using oligo-(dT) primer and reverse transcriptase (Invitrogen). Primers used for the amplification of murine C3, Caspase 3 and GAPDH were as follows: C3, 5′-CCCTgCCCCTTACCCCTTCATTC-3′ (forward), and 5′-CGTACTTGTGCCCCTCCTTA TCTG-3′ (reverse); Caspase 3, 5′-CGGGGTACGGAGCTGGACTGT-3′ (forward) and 5′-AATTCCGTTGCCACCTTCCTGTT-3′ (reverse), and GAPDH, 5′-tgatgacatcaagaag gtggtgaa-3′ (forward) and 5′-tgggatggaaattgtgagg gagat-3′ (reverse). PCR reactions were performed under the following conditions: 95° C. for 30 sec, 58° C. for 30 sec, and then 72° C. for 30 sec (30 cycles).

Real-time PCR reactions were performed using SYBR Green PCR Master mix (Stratagene) and 100 nM of gene-specific forward and reverse primers with the same sequences as RT-PCR. The PCR reaction conditions were 95° C. for 10 min, 95° C. for 30 sec, 58° C. for 1 min and 72° C. for 30 sec (40 cycles).

### Statistical Analysis

Data are expressed as means ±SEM. Statistical comparisons among groups were performed using Student's t test. Statistical significance was determined as p<0.05.

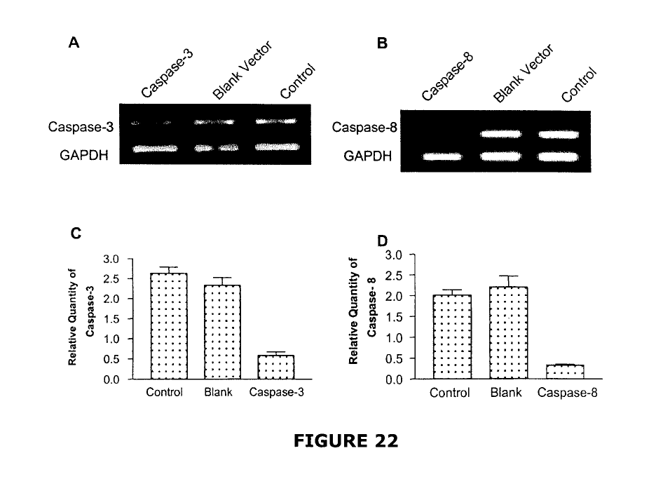

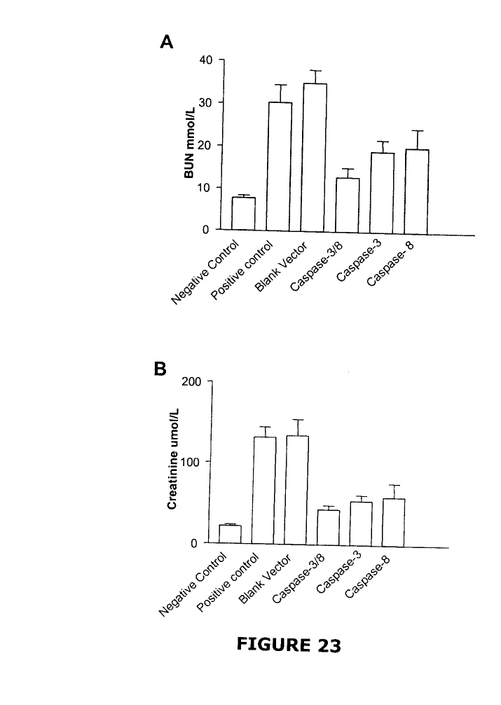

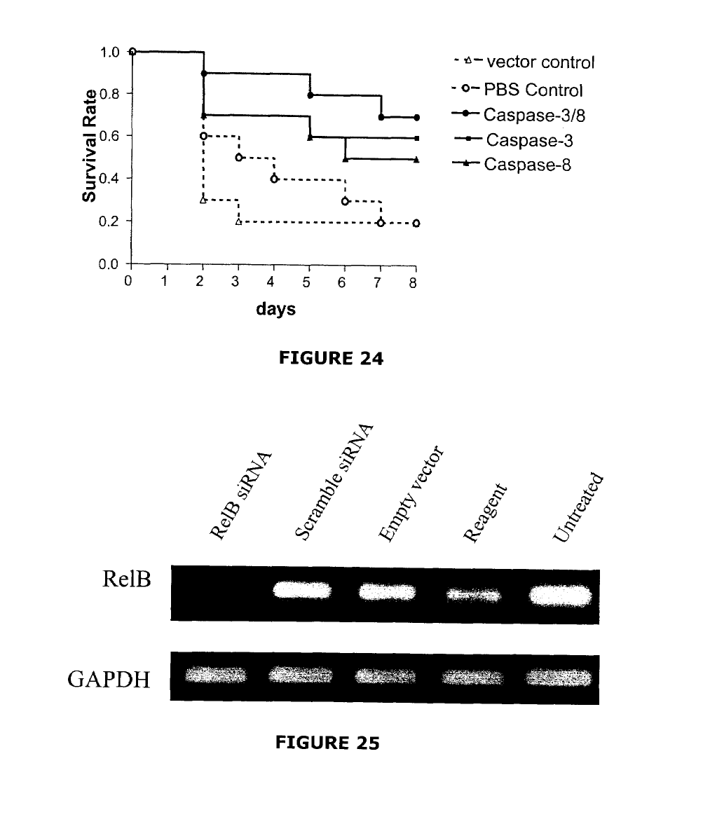

### General Methods Used (FIGS. 21-24)

Caspase-3 and Caspase-8 siRNA Design

Target sequences of caspase-3 gene and of caspase-8 gene were selected. The oligonucleotides containing sequences specific for caspase-3 (sense 5′GATCCCAACTACTGTCCAGATAGATCCTTGATATCCGGGATCTATCTGGACAGTAG TTTTTTTTCCAAA-3′, antisense 5′-AGCTTTTGGAAAAAA AACTACTGTCCAGATAGATCCTCTCTTGAAGGATCTATCTGGACAGTAGTTGG-3′) and for caspase-8 (5′-GATCCGACCTTTAAGGAGCTTCATTTCAAGAGA ATGAAGCTCCTTAAAGGTCTTTTTTGGAAA-3′, antisense 5′-AGCTTTTCCAAAAAAGACCTTTAAGGAGCTTCATTCTCTTGAA ATGAAGCTCCTTAAAGGTCG-3′) were synthesized and annealed. Caspase-3 siRNA and caspase-8 siRNA vectors that expressed hairpin siRNAs under the control of the mouse U6 promoter and cGFP genes were constructed, by inserting pairs of annealed DNA oligonucleotides into a pRNAT-U6.1/Neo siRNA expression vector that had been digested with Bam HI and Hind III (Genescript, Piscataway, N.J.).

### In Vitro Silencing of the Caspase-3 and Caspase-8 Gene

L929 cells were transfected with caspase-3/8 siRNA using lipofectamine 2000. The vehicle alone and scrambled (nonsense) siRNA were used as negative controls. Briefly, cells were plated into 24-well plates (1×10⁵ of cells per well) and allowed to grow overnight, to reach 90% confluence. Cells were transfected with 2 μg caspase-3/8 siRNA or negative control siRNA plasmids in serum-reduced medium for 5 hr, then incubated in complete medium for 24 hr. All RNA was prepared for subsequent analysis.

### RelB SiRNA Preparation

RelB siRNA oligos were synthesized by Welgen, Inc. The siRNA was cloned into the pQuiet vector by the restricted site.

### In Vitro Silencing of the RelB Gene

The L929 cell line was transfected with RelB siRNA using Lipofectamine 2000 (Invitrogen). The vehicle alone and scrambled (nonsense) siRNA were used as negative controls. Briefly, after culture for 24 hours, TRIzol was used to extract RNA for reverse transcriptase PCR (RT-PCR).

### Animals for In Vivo Study

CD1 mice were purchased from The Jackson Laboratory (Bar Harbor, Me.). The mice were maintained under specific pathogen-free conditions. All mice were male and 6 to 10 weeks old. All experiments were performed in accordance with the Guide for the Care and Use on Animals Committee Guidelines.

### SiRNA Injections

Forty-eight hours before renal IRI, siRNAs (50 μg in 1 ml of PBS) or 1 ml of PBS was rapidly injected (within 10 sec) into one of the tail side veins. To dilate tail veins, the tail was immersed in warm water or warmed by lamp.

### Renal IRI Model

Mice weighing 25-27 g were anesthetized with an intraperitoneal injection of Ketamine (100 mg/kg) and Xylazine (10 g/kg) and placed on a heating pad to maintain their body temperature during surgery. Following abdominal incisions, renal pedicles were bluntly dissected and a microvascular clamp (Roboz Surgical Instrument, Washington, D.C.) was placed on the left renal pedicle for 25 min. During the procedure, animals were kept well-hydrated with warm saline and at a constant temperature (37° C.). The time of ischemia, 25 min, was chosen to obtain a reversible model of ischemic ARF with a minimum of vascular thrombosis, and to avoid animal mortality. After removal of the clamp, the left kidney was observed for an additional 1 min to see the color change indicative of blood reflow, then the right kidney was resected. Thereafter, incisions were sutured, and the animals were allowed to recover, with free access to food and water. Blood was collected were collected from inferior vena cava and the left kidney was harvested for analysis 24 hr after reperfusion. For the survival observance experiment, surgery was performed in an identical fashion, except that the time of ischemia (clamping) was 35 min.

### Assessment of Renal Function

Blood samples were obtained from the inferior vena cava (pre-ischemia) and at 24 h post-ischemia. Blood Urea Nitrogen (BUN) and Serum Cretinine (Cr) levels were measured by the core laboratory at the London Health Sciences Center using SYNCHRON LX Systems (Beckman Coulter, Inc.).

Measurement of Renal RelB mRNA Levels by Quantitative Real Time PCR

Total RNA was isolated from cells or kidney by using TRIzol (Life Technologies, Gaithersburg, Md.). Primers for RelB RT-PCR were: sense CCC CTA CAA TGC TGG CTC CCT GAA, antisense CAC GGC CCG CTC TCC TTG TTG ATT. RT-PCR was performed by using an eppendorf Cycler (Mastercycler gradient). All reactions were done in a 50-pI reaction volume, following the manufacturer's instructions. PCR parameters consisted of 50 min of reverse transcription at 42° C., followed by 30 cycles of PCR at 94° C. for 30 sec, 58° C. for 30 sec, and 72° C. for 30 sec. Gel electrophoresis was run by 100 voltages and then the results observed under UV light. Also samples were done by real-time PCR for the gene expression.

### Assessment of Renal Morphological Changes

At 24 h post-ischemia, kidneys were dissected from mice and tissue slices were fixed in 10% Formalin and processed for histology examination using standard techniques. Formalin tissue was embedded in paraffin and 5-Lm sections were stained with H&E. These sections were examined in a blinded fashion by a pathologist. The percentage of histology changes in the cortex and medulla were scored using a semi-quantitative scale designed to evaluate the degree of infarction, tubular vasculization, and cast formation on a five-point scale based on injury area of involvement as follows: 0, normal kidney; 0.5, <10%; 1, 10-25%; 2, 25-50%; 3, 50-75%; and 4, 75-100%. A pathologist quantitatively assessed neutrophil infiltration by counting the number of neutrophils per high powered field (×400) over five fields, then averaging neutrophil numbers.

### Statistical Analysis

Statistical comparison was by two-sided Student's t test. Survival was analyzed by Kaplan-Meier test.

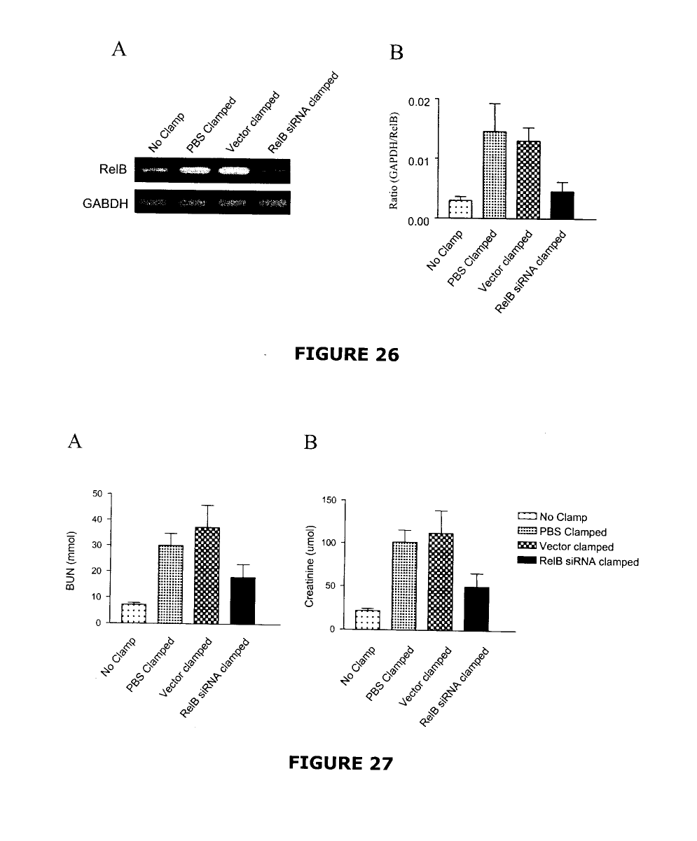

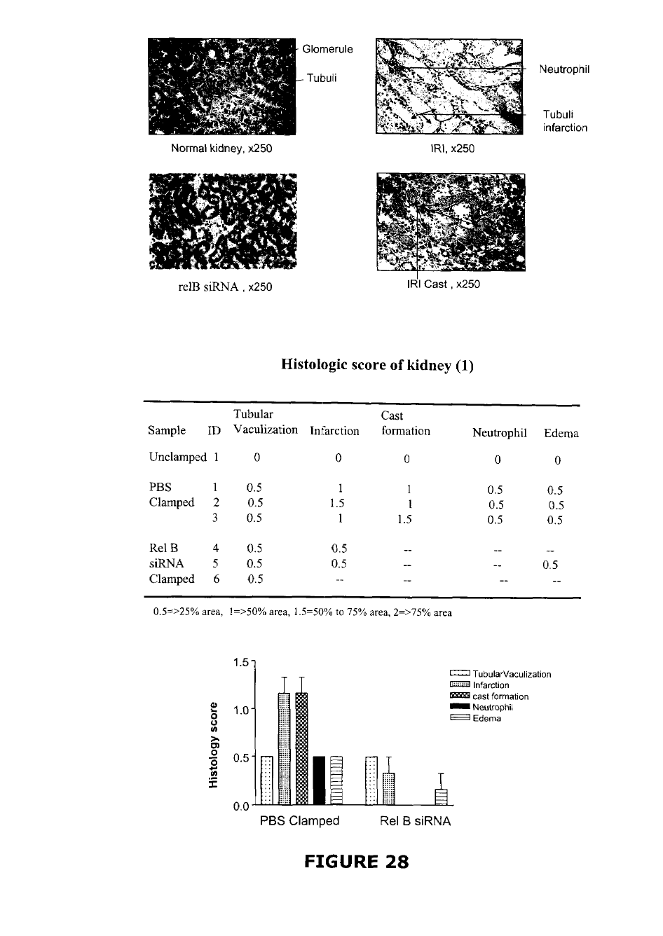

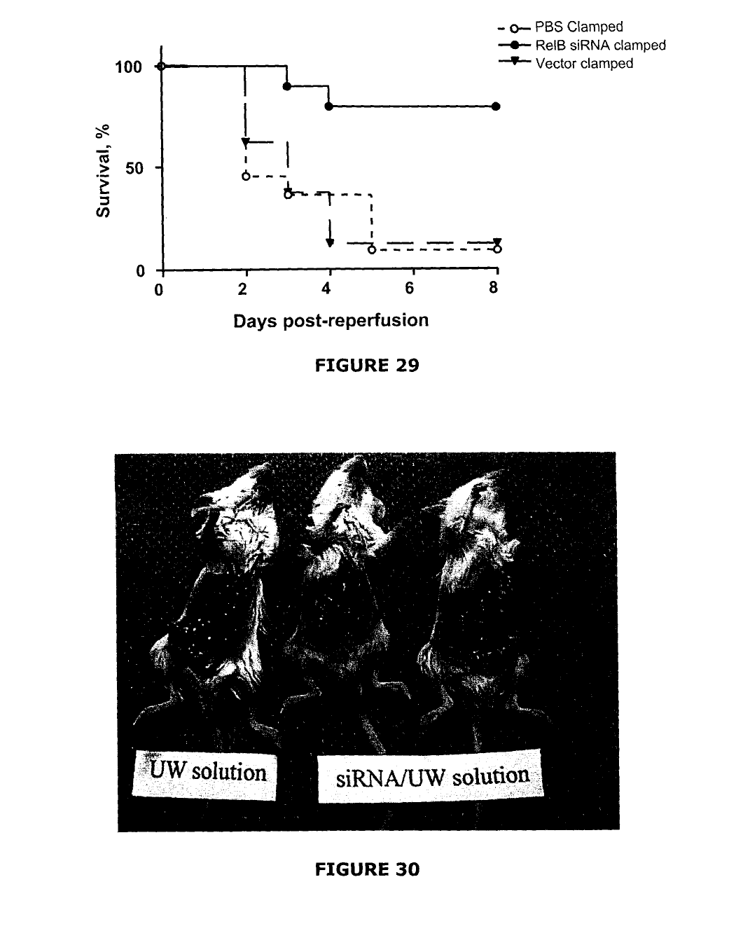

### General Methods Used (FIGS. 25-29)

### Animals

Six-week-old male CD1 (Charles River) mice weighing 25-27 g were maintained under our university facility till to use. All procedures were performed in accordance with guidelines set by the Guide for the Care and Use on Animals Committee.

### Cell Lines

The cell lines, L929 and Macrophage, were used in our experiment for detect the silence efficiency of siRNA. To assay the silence efficacy of siRNA, we transfected the siRNA into L929 cell line by using Lipofectamine 2000 (Invitrogen), then tTRIzol extracted the RNA for reverse transcriptase PCR (RT-PCR).

SiRNA Preparation and Injection

TNFα siRNA was synthesized by Company (Invitrogen). This TNFα siRNA was constructed into the pSilence vector or pRNAT U6.1 vector. For hydrodynamic injection, siRNAs (50 μg in 1 ml of PBS) or 1 ml of PBS was rapidly injected (within 10 sec) into one of the tail side veins. To dilate tail veins, the tail was immersed in warm water or warmed by lamp.

### Ischemia Protocol

Mice (25-27 g) were anesthetized by intraperitoneal administration of Ketamine/Xylazine (100 mg/kg and 10 g/kg) Body temperature was kept constant by placing a warm pad beneath the animal. Using a midline abdominal incision, the left renal arteries and veins were occluded for 25 or 35 minutes with microaneurysm clamps (International Fine Science Tools, Inc. CA), while the right kidney was removed. After occlusion, 0.5 ml of prewarmed (37° C.) saline was placed in the abdominal cavity and the abdomen was closed. After removal of the clams, the kidneys were observed for an additional 1 minute to see the color change indicative of blood reflow. After suturing, the incision mice were returned to their cages. Sham-treated mice had identical surgical procedures except that microaneurysm clamps were not applied. Mice were sacrificed at 24 hours; blood samples were collected by inferior vena cava. For survival experiments, animals were observed for several days after all surviving animals were free of signs of illness.

### Assessment of Renal Function

Blood Urea Nitrogen (BUN) and Cretinine (Cr) were measured from serum by our core facility method using SYNCHRON LX Systems (Beckman Coulter, Inc.).

### RT-PCR

Total RNA was isolated from cells or kidney by using TRIzol (Life Technologies, Gaithersburg, Md.). RT-PCR was performed by using an eppendorf Cycler (Mastercycler gradient). All reactions were done in a 50-μl reaction volume, following the manufacturer's instructions. PCR parameters consisted of 50 min of reverse transcription at 42° C., followed by 30 cycles of PCR at 94° C. for 30 sec, 58° C. for 30 sec, and 72° C. for 30 sec. Gel electrophoresis was run by 100 voltages and then the results observed under UV light.

### Assessment of Renal Morphological Changes

After surgical removal from mice, kidneys were cut coronally, fixed in 10% formaldehyde and embedded in paraffin. Sections (5 um) were stained with H&E. One whole deep coronal section was examined under a microscope. The percentage of tubules damaged in the corticomedullary junction was estimated using a five-point scale

### RelB Immunohistochemistry

After deparaffinization and rehydration, paraffin sections of the kidneys were incubated with 3% hydrogen peroxide for 15 min to quench endogenous peroxidase activity. After microwaving for 20 min, sections were blocked for 30 min in wash buffer containing 5% normal mouse serum. Sections were incubated for 1 h at room temperature with hamster anti-mouse Fas mAb (BD Pharmingen) diluted 1:100 in PBS. After washing with PBS, sections were incubated with biotinylated mouse anti-hamster Ig and then with streptavidin conjugated with horseradish peroxidase (LSAB detection kit, DAKO). After further washes in PBS, staining was developed with diaminobenzidine (DAB), and slides were lightly counter-stained with hematoxylin. Control slides were stained with hamster IgG replacing primary antibody. Fas immunostaining appears in all or none of the epithelial cells in individual renal tubules. The percentage of positive tubules in five consecutive fields of view (magnification, ×200) was assessed in a blinded manner.

### Histologic Score

Kidneys were removed after kidney ischemia and fixed in 10% formalin for histological examination. Tissues were embedded in paraffin and sections were stained with H&E. The mean was calculated from the blinded analysis by using a score of 0, no damage; 0.5, <10%; 1, 10-25%; 2, 25-50%; 3, 50-75%; and 4, 75-100%. Neutrophile filtration was assessed by counting the number of neutrophile cells over five fields (×400).

### Statistical Analysis

Statistical comparison was by two-sided Student's t test. Survival was analyzed by Kaplan-Meier test.

### General Protocols for TNF-Alpha

TNFα Gene Silenced by relB siRNA

To observe the silence results of gene in vitro, L929 cells were transfected by vector relB siRNA. Twenty-four hours later, the cells were collected and RNA was prepared. Expression of relB mRNA in L929 was reduced as determined by RT-PCR. Next it was determined whether TNFα siRNA injection could silence up-regulated TNFα expression after ischemia reperfusion damage. Expression of TNFα after IRI was first observed. Expression of TNFα was increased after IRI for 24 hours and 48 hours, the highest expression is at 48 hours in kidney. Vector siRNA (50 μg) was then delivered by a single hydrodynamic injection into the tail vein. After 24 hours and 48 hours, the kidneys were taken and make homogenates. RNA and cDNA were prepared and RT-PCR was performed. The results showed that the expression of relB was decreased after treated with siRNA.

Kidney Function Improved by siRNA Treatment

The results of gene silence indicated that siRNA could block the pathway of NF-kB. It was demonstrated that TNFα could prevent the damage of kidney after ischemia. BUN and creatinine were measured by our core facility. After 25 minutes clip, the level of BUN and creatinine were obviously increased compared with untreated control mice. BUN level decreased 30% if compared with positive control group. However creatinine contents decreased significantly.

### Morphological Changes

Next the change of kidney pathology was observed. Renal pathology was determined by H&E (hematoxylin) staining was significantly reduced in the ischemic kidney with silenced TNFα expression.

Mice mortality was improved due to the fact that TNFα expression in the kidney and ischemic damage were suppressed. It was next demonstrated that TNFα siRNA could provide protection from critical ischemia in mice, which the left renal pedicle was clamped for 35 minutes and the contralateral kidney was removed. Before clamping, the mice were injected saline, vector siRNA and TNFα siRNA before 48 hours by hydrodynamic tail vein injection. Ten of 11 mice that received saline by hydrodynamic tail vein injection died of acute renal failure within in 5 days. However, 8 of 10 mice injected with TNFα siRNA survived (P<0.01 vs empty vector, P<0.01 vs saline).

### EXAMPLES

### FIGS. 1-16

### Example 1

CD1 mice were subjected to clamping of the left renal vein and artery, as described above, for 25 min. at 37° C. The treated kidney was removed after 24 or 48 hours of reperfusion. The time points are measured from the time of clamping. The total RNA was extracted from IDO-silenced, nonsense-siRNA-silenced, or mock-transfected B16F10 cells was isolated using TRIzol reagent (Gibco BRL) according to the manufacturer's instructions. RelB gene expression was determined by RT-PCR First strand cDNA was synthesized using an RNA PCR kit (Gibco BRL) with the supplied oligo d(T)16 primer. One μmol of reverse transcription reaction product was used for the subsequent PCR reaction. The primers used for RelB flanked the RelB-siRNA target sequences, and GAPDH (internal negative control) primers were used. PCR conditions used were as follows: 94° C. for 30 s, 58° C. for 30 s, and 72° C. for 30 s (30 cycles). PCR products were visualized using gel electrophoresis by staining with ethidium bromide in a 1.5% agarose gel (Hill, J. A., Ichim, T. E., Kusznieruk, K. P., Li, M., Huang, X., Yan, X., Zhong, R., Cairns, E., Bell, D. A., and Min, W. P. 2003. Immune modulation by silencing IL-12 production in dendritic cells using small interfering RNA. J Immunol 171:691-696). As seen in FIG. 1, RelB expression was increased after kidney ischemia.

### Example 2

CD1 mice were treated as described in Example 1 and Fas gene expression in the treated kidney was determined by RT-PCR after 0, 2, 12 and 24 hours of reperfusion. As seen in FIG. 2, Fas expression was increased after ischemia.

### Example 3

CD1 mice were treated as described in Example 1 and caspase 8 gene expression in the treated kidney was determined after 0, 2, 12, 24 or 48 hours of reperfusion. As seen in FIG. 3, caspase 8 expression was increased after ischemia.

### Example 4

CD1 mice were treated as described in Example 1 and caspase 3 and GAPDH gene expression was determined. FIG. 4 shows the ratio of caspase 3:GAPDH expression at 0, 2, and 24 hours after reperfusion.

### Example 5

CD1 mice were treated as described in Example 1 and C3 gene expression in the treated kidney was determined after 24 and 48 hours of reperfusion. FIG. 5 shows that C3 gene expression was increased after ischemia.

### Example 6

CD1 mice were treated as described in Example 1 and C5aR gene expression in the treated kidney was determined after 24 and 48 hours of reperfusion. FIG. 6 shows that C5aR expression was increased after ischemia.

### Example 7

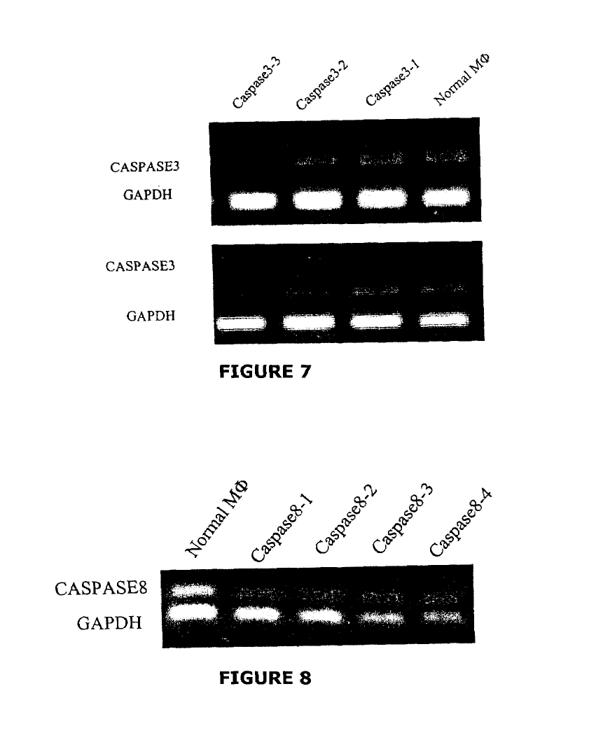

FIG. 7 shows silencing Caspase 3 gene in vitro in a macrophage cell line. Briefly, cells were plated into either 12-well plates (2×10⁵ cells per well) and allowed to grow overnight in 1 or 2 ml of complete medium without antibiotics. Four μg of Caspase 3-siRNA-containing plasmid was incubated with 10 μl of Lipofectamine 2000 reagent in 250 μl of Optimal serum-reduced medium (Invitrogen) at room temperature for 20 min. The mixture was then added to cell cultures grown to 90%-95% confluence. After 4 hrs of incubation an equal volume of RPMI 1640 supplemented with 20% FCS. Twenty four to 48 h later, transfected cells were harvested to detect Caspase gene expression by RT-PCR. (MØ=Macrophages)

### Example 8

Macrophage cells as in Example 7 were transfected with caspase 8-siRNA-expressing vectors as described in Example 7. 48 hours after transfection, total RNA was extracted and caspase 8 gene expression was determined by RT-PCR. 1, 2, 3 and 4 represent different siRNA. FIG. 8 shows that caspase 8 expression was reduced in the treated cells.

### Example 9

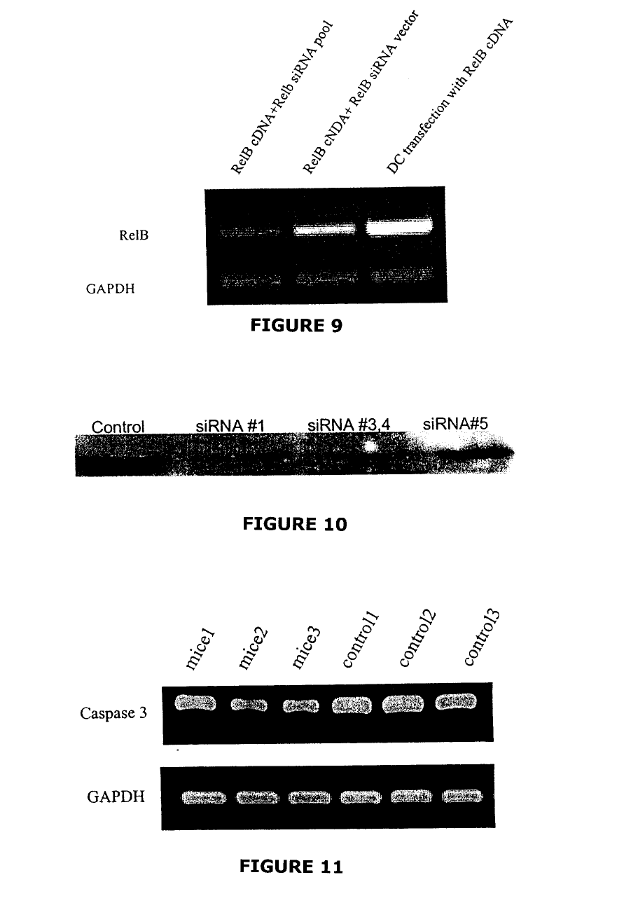

Macrophage cells as in Example 7 were co-transfected, by the method described in Example 7, with Rel B cDNA and Rel B-siRNA. 48 hours after transfection, total RNA was extracted and Rel B gene expression was determined by RT-PCR. The siRNA pool is a mixture of several siRNA that targets the same gene but different sites and siRNA vector is a vector containing a SEC sequence or a hair pin siRNA sequence as is described in the application. All lanes are DC cells. As seen in FIG. 9, RelB expression was reduced by siRNA pool and siRNA vector.

### Example 10

Macrophage cells which express Fas were transfected as described above with Fas-siRNA in a Fas-siRNA-expressing vector. 48 hours after transfection, protein was extracted and Fas expression was determined by Western blot. As seen in FIG. 10 protein levels were reduced in Fas-siRNA treated cells. siRNA 1, 3, 4 and 5 are different siRNA sequences that target the Fas gene.

### Example 11

CD1 Mice were injected intravenously with 50 μg siRNA specific for caspase 3 gene. The left renal vein and artery were then clamped as described in Example 1. 48 hours after siRNA injection, the left kidney was harvested, total RNA was extracted and caspase 3 expression was determined by RT-PCR.

### Example 12

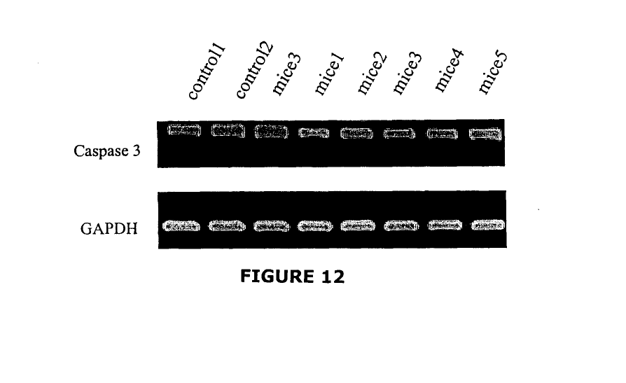

CD1 mice were treated as described in Example 11, except that siRNA specific for the Fas gene was injected. FIG. 12 shows that Fas gene expression was reduced after siRNA treatment. From the left to the right, Control 1-3, and mice 1-5.

### Example 13

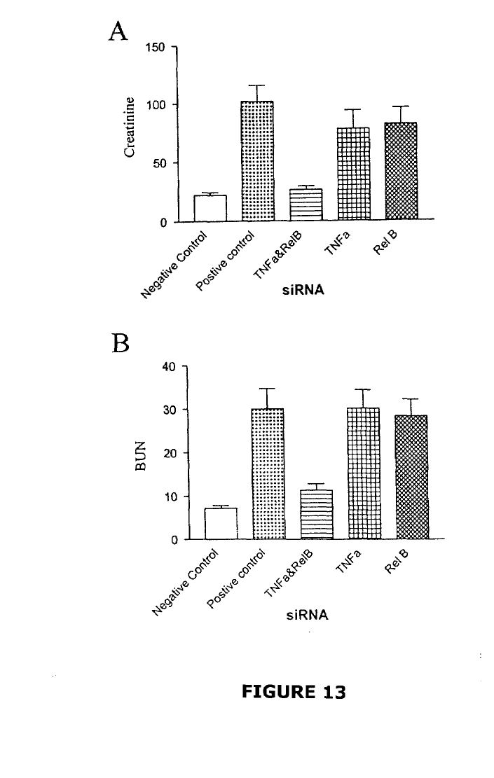

CD1 mice were injected i.v. with siRNA specific for TNFα and/or Rel B genes (50 μg of each siRNA in combinations) and subjected to left kidney ischemia as described in Example 1. Renal function was determined by measuring blood creatinine and BUN levels 24 hours after reperfusion. FIG. 13 shows that renal function was preserved at close to normal after ischemia when siRNA to both TNFα and Rel B genes was used.

### Example 14

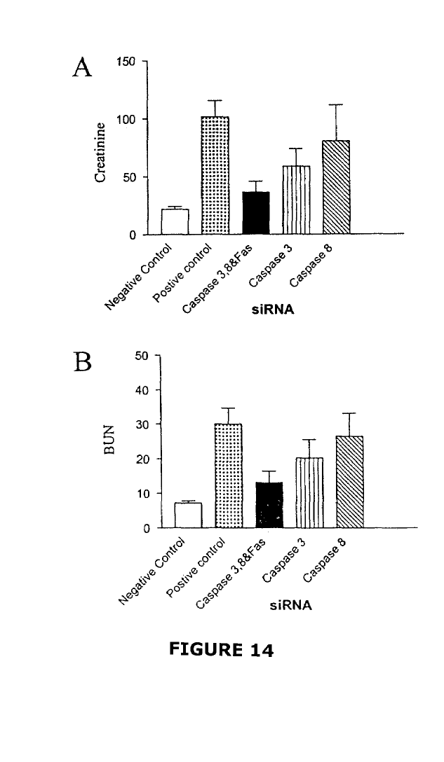

CD1 mice were injected i.v. with siRNA specific for the apoptotic genes caspase 3, caspase 8 and Fas, either separately or in combination, and subjected to left kidney ischemia as described in Example 1. Renal function was determined 24 hours after reperfusion. As seen in FIG. 14, silencing of caspase 3 or caspase 8 alone protected renal function after ischemia. Most effective was use of a mixture of siRNAs targeted to caspase 3, caspase 8 and Fas.

### Example 15

CD1 mice were treated as described in Example 13, except for use of siRNA in the following combinations:

| | |
|---|---|
| Combination 1: | caspase 3, caspase 8 and Fas; |
| Combination 2: | TNFα and Rel B; |
| Combination 3: | C3 and C5aR; |
| Combination 4 (S-mix): | all of the siRNAs of mixtures 1, 2 and 3. |

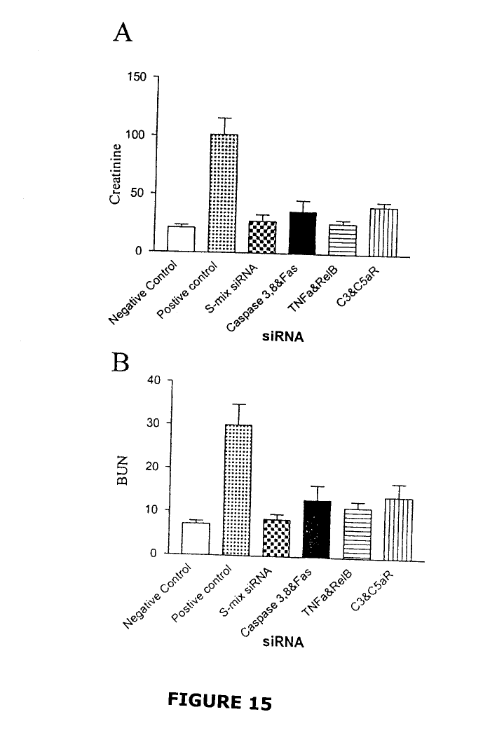

FIG. 15 shows good protection of renal function after ischemia by all of the siRNA combinations.

### Example 16

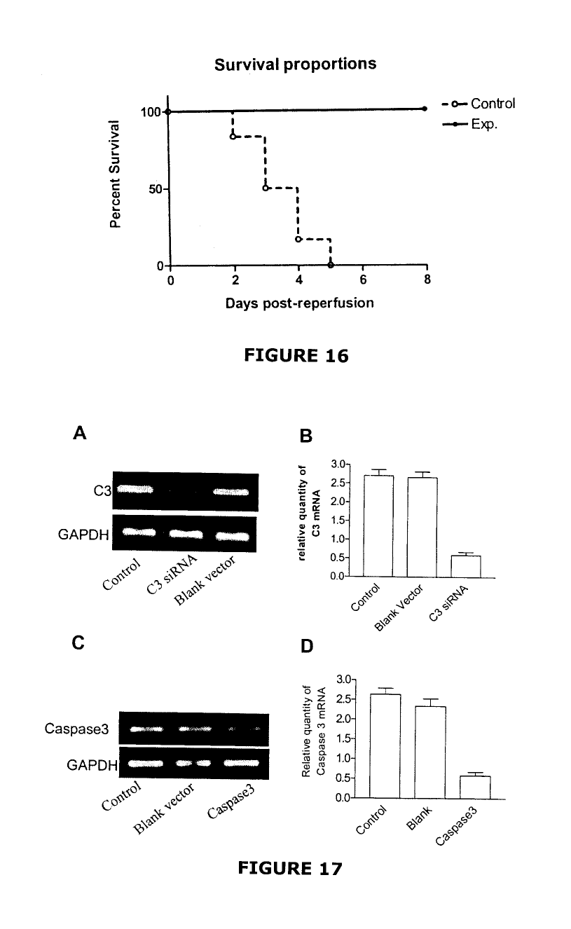

CD1 mice were injected i.v. with 50 μg of a siRNA mixture specific for caspase 3, caspase 8, Fas, C3, C5aR, TNFα and Rel B genes or with saline (controls) and subjected to left renal ischemia by clamping of the renal vein and artery for 35 min. at 37° C. Survival of the mice was followed after reperfusion and the results are shown in FIG. 16. All siRNA-treated mice were alive 8 days after reperfusion, whereas all control mice had died by 5 days after reperfusion.

Although preferred embodiments of the invention have been described herein in detail, it will be understood by those skilled in the art that variations may be made thereto without departing from the spirit of the invention or the scope of the appended claims.

1. A composition for maintaining cells, tissues and/or organs in a viable state ex vivo during storage and in vivo during reperfusion, the composition comprising siRNA specific for genes whose expression is associated with loss of viability or cell damage in ex vivo tissues or organs.

2. The composition of claim 1, wherein said genes are selected from the group consisting of apoptosis genes, immuno-inflammatory genes, complement genes and combinations thereof.

3. The composition of claim 2, wherein said apoptosis genes are selected from the group consisting of Caspase 3, Caspase 8, fas and combinations thereof.

4. The composition of claim 2, wherein said immuno-inflammatory genes are selected from the group consisting of TNF-alpha, RelB and combinations thereof.

5. The composition of claim 2, wherein said complement genes are selected from the group consisting of C3, C5aR and combinations thereof.

6. The composition of claim 2, wherein said composition comprises siRNA specific for Caspase 3, Caspase 8, Fas, C3, C5aR, TNF-alpha, and RelB.

7. The composition of claim 2, wherein said composition comprises siRNA specific for caspase 3, caspase 8 and Fas.

8. The composition of claim 2, wherein said composition comprises siRNA specific for TNFα and RelB.

9. The composition of claim 2, wherein said composition comprises siRNA specific for C3 and C5aR.

10. The composition of claim 1, wherein said composition is provided at temperatures of about 4° C. up to about 37° C.

11. The composition of claim 10, wherein said composition is provided at 37° C.

12. The composition of claim 1, wherein said composition further comprises a commercially available organ storage solution.

13. The composition of claim 1, wherein said composition further comprises additives selected from the group consisting of free radical scavengers, lazaroids, anti-apoptosis agents, calcium channel blockers, intercellular adhesion molecule-1 inhibitors, pentoxifylline and combinations thereof.

14. The composition of claim 1, wherein said siRNA is provided in amounts of up to about 100 μg/ml.

15. A method for maintaining the viability of a tissue, cells or an organ maintained ex vivo prior to transplantation, the method comprising contacting the tissue, cells or organ with at least one siRNA specific for a gene whose expression is associated with loss of viability or cell damage in ex vivo tissues, cells or organs, wherein said contact is made at temperatures of over about 4° C.

16. A method for protecting a tissue, cells or an organ of a mammal against ischemic and/or reperfusion injury comprising contacting the tissue, cells or organ with at least one siRNA specific for a gene whose expression is associated with ischemic and/or reperfusion injury.

17. The method of claims 15 or 16, wherein said at least one siRNA is specific for genes selected from the group consisting of apoptosis genes, immuno-inflammatory genes, complement genes and combinations thereof.

18. The method of claim 17, wherein said apoptosis genes are selected from the group consisting of caspase-3, caspase-8, fas and combinations thereof.

19. The method of claim 17, wherein said immuno-inflammatory genes are selected from the group consisting of TNF-alpha, RelB and combinations thereof.

20. The method of claim 17, wherein said complement genes are selected from the group consisting of C3, C5aR and combinations thereof.

21. The method of claim 17, wherein said siRNA genes used are selected from the group consisting of:
(a) Caspase 3, Caspase 8, Fas, C3, C5aR, TNF-alpha, and RelB;
(b) caspase 3, caspase 8 and Fas;
(c) TNFα and RelB; and
(d) C3 and C5aR.

22.-24. (canceled)

25. The method of claim 17, wherein said siRNA is provided in amounts of up to about 100 μg/ml for each of said siRNA.

26. A method of storage of a cell, tissue or organ in a viable state, the method comprising:
i) contacting a cell, tissue or organ to be stored with a composition comprising at least one siRNA specific for a gene whose expression is associated with loss of viability or cell damage; and
ii) maintaining the cell, tissue or organ in contact with the solution at temperatures of from about sub-ambient up to about 37° C.

27. The method of claim 26, wherein a combination of siRNA is used.

28. The method of 26, wherein said gene is selected from the group consisting of apoptosis genes, immuno-inflammatory genes, complement genes and combinations thereof.

29. The method of claim 28, wherein said apoptosis genes are selected from the group consisting of caspase-3, caspase-8, fas and combinations thereof.

30. The method of claim 28, wherein said immuno-inflammatory genes are selected from the group consisting of TNF-alpha, RelB and combinations thereof.

31. The method of claim 28, wherein said complement genes are selected from the group consisting of C3, C5aR and combinations thereof.

32. The method of claim 28, wherein said siRNA genes used are selected from the group consisting of:
(a) Caspase 3, Caspase 8, Fas, C3, C5aR, TNF-alpha, and RelB;
(b) caspase 3, caspase 8 and Fas:
(c) TNFα and RelB: and
(d) C3 and C5aR.

33.-35. (canceled)

36. The method of claim 28 wherein said siRNA is provided in amounts of up to about 100 μg/ml for each of said siRNA.

37. The method of claim 28, wherein said method is performed at temperatures of up to about 37° C.

38. The method of claim 28, wherein said composition further comprises a commercially available organ storage solution.

39. The method of claim 26 wherein, said composition further comprises additives selected from the group consisting of free radical scavengers, lazaroids, anti-apoptosis agents, calcium channel blockers, intercellular adhesion molecule-1 inhibitors, pentoxifylline and combinations thereof.

40. An ex vivo altered cell, tissue or organ made by the composition of claim 1, wherein said altered cell, tissue or organ comprises a combination of siRNA targeted to silence the expression of one or more apoptosis genes, immuno-inflammatory genes and complement genes.

41. The cell, tissue and/or organ of claim 40, wherein said apoptosis genes are selected from the group consisting of Caspase 3, Caspase 8, fas and combinations thereof.

42. The cell, tissue and/or organ of claim 40, wherein said immuno-inflammatory genes are selected from the group consisting of TNF-alpha, RelB and combinations thereof.

43. The cell, tissue and/or organ of claim 40, wherein said complement genes are selected from the group consisting of C3, C5aR and combinations thereof.

44. The cell, tissue and/or organ of claim 40, wherein said siRNA genes are selected from the group consisting of:
(a) Caspase 3, Caspase 8, Fas, C3, C5aR, TNF-alpha, and RelB;
(b) Caspase 3, Caspase 8 and Fas;
(c) TNFα and RelB; and
(d) C3 and C5aR.

45-47. (canceled)

48. The method of claim 17 or 26, wherein said organ is selected from the group consisting of heart, lung, kidney, liver and pancreas.

49. The method of claim 48, wherein said organ is kidney.

50. The composition of claim 1, wherein the cell, tissue and/or organ is selected from the group consisting of heart, lung, kidney, liver and pancreas.
